(19) United States
(12) Patent Application Publication
(10) Pub. No.: US 2003/0139366 A1
(43) Pub. Date: Jul. 24, 2003

(54) Inhibition of Smad3 to prevent fibrosis and improve wound healing

(76) Inventors: Anita B. Roberts, Bethesda, MD (US); Gillian S. Ashcroft, Lancashire (GB); Angelo Russo, Bethesda, MD (US); James B. Mitchell, Damascus, MD (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/299,886

(22) Filed: Nov. 18, 2002

Related U.S. Application Data
(63) Continuation of application No. PCT/US00/13725, filed on May 19, 2000.

(51) Int. Cl. A61K 48/00; A61K 38/17

(52) U.S. Cl. 514/44; 514/12

(57) ABSTRACT

The invention is related to modulation of Smad3 expression to prevent fibrosis and improve wound healing. Aspects of the invention, for example, include approaches to improve wound healing and/or reduce or prevent fibrosis by inhibiting Smad3. Embodiments described herein also include approaches to identify componds that modulate Smad3 expression and the preparation of pharmaceuticals comprising said compounds.

### CROSS-REFERENCE TO RELATED APPLICATIONS

This application is a continuation of international application number PCT/US00/13725, and claims the benefit of priority of international application number PCT/US00/13725 having international filing date of May 19, 2000, designating the United States of America and published in English, which is hereby expressly incorporated by reference in its entirety.

### BACKGROUND OF THE INVENTION

1. Field of the Invention

The invention is related to inhibition of Smad3 to prevent fibrosis and improve wound healing.

2. Description of the Related Art

Both Smad3 and its closely related homologue, Smad2, are intracellular mediators of TGF-β function, acting as nuclear transcriptional activators (Massague, J. TGF-beta signal transduction. Annu. Rev. Biochem. 67, 753-791 (1998)); (Derynck, R., Zhang Y. & Feng, X. H. Smads: transcriptional activators of TGF-beta responses. Cell 95, 737-740 (1998)). Smad2 and Smad3 mediate intracellular signaling from TGF-βs 1, 2, 3 and activin, each of which has been implicated as an important factor in the cellular proliferation, differentiation and migration pivotal to cutaneous wound healing (Roberts, A. B. TGF-beta: activity and efficacy in animal models of wound healing. Wound Repair Regen. 3, 408-418 (1995)); (O'Kane, S. & Ferguson, M. W. 3. TGF-beta s and wound healing. Int. J. Biochem. Cell Biol. 29, 63-78 (1997)). Mice null for Smad3 (Smad3^{ex8/ex8} mice) survive into adulthood, unlike Smad2-null mice which do not survive embryogenesis (Yang, X. et al. Targeted disruption of SMAD3 results in impaired mucosal immunity and diminished T cell responsiveness to TGF-beta. EMBO J. 188, 1280-1291 (1999)); (Datto, M. B. et al. Targeted disruption of Smad3 reveals an essential role in transforming growth factor beta-mediated signal transduction. Mol. Cell Biol. 19, 2495-2504 (1999)); (Zhu, Y., Richardson, J. A., Parada, L. F., & Graff, J. M. Smad3 mutant mice develop metastatic colorectal cancer. Cell 18, 703-714 (1998)); (Weinstein, M., Yang, X., Li, C., Xu, X., & Deng, C. Failure of extraembryonic membrane formation and mesoderm induction in embryos lacking the tumor suppressor Smad2. Proc. Natl Acad. Sci. USA 95, 9378-9383 (1998)). Here, to identify selective targets of Smad3 signaling pathways in vivo, we studied its role in cutaneous wound healing using wild-type mice or mice heterozygous or null for the Smad3 gene following targeted disruption (Yang, X. et al. Targeted disruption of SMAD3 results in impaired mucosal immunity and diminished T cell responsiveness to TGF-beta. EMBO J. 188, 1280-1291 (1999)).

### SUMMARY OF THE INVENTION

The generation of animals lacking SMAD proteins, which transduce signals from transforming growth factor-β (TGF-β), has made it possible to explore the contribution of the SMAD proteins to TGF-β activity in vivo. Here we report that, in contrast to predictions made on the basis of the ability of exogenous TGF-β to improve wound healing, Smad3-null (Smad3^{ex8/ex8}) mice paradoxically showed accelerated cutaneous wound healing compared with wild-type mice, characterized by an increased rate of re-epithelialization and significantly reduced local infiltration of monocytes. Smad3^{ex8/ex8} keratinocytes showed altered patterns of growth and migration, and Smad3^{ex8/ex8} monocytes exhibited a selectively blunted chemotactic response to TGF-β. These data provide evidence that Smad3 is involved in specific pathways of tissue repair and in the modulation of keratinocyte and monocyte function in vivo.

### BRIEF DESCRIPTION OF THE DRAWINGS

FIG. A: Proposed suppression of endogenous Smad3 to improve wound healing. Chronic wounds are characterized by delayed re-epithelialization and increased inflammation. Application of TGF-β to these wounds impairs healing further by inhibiting keratinocyte proliferation and stimulating monocyte and neutrophil recruitment. Conversely, treatment of chronic wounds with agents that suppress Smad3 expression would be predicted to stimulate re-epithelialization, inhibit inflammation, and reduce local levels of TGF-β. Subsequent application of exogenous TGF-β to such wounds would stimulate matrix deposition via Smad3-independent pathways, but would have no impact on the Smad3-dependent pathways.

FIG. 1: Accelerated wound healing in Smad3-null mice is associated with a reduced monocytic response. a, Wound areas were determined using image analysis. Results are means±s.e.m., n=10 for each time point and group. *P<0.05 compared with wild-type (Student's t-test). d, day. b, Re-epithelialization was determined as the percentage of distance migrated by the neo-epidermis compared with the upper wound width. Results are means±s.e.m., n=10 for each time point and group. *P<0.05 compared with wild-type. S2 HT, Smad2 heterozygotes. c, Cell numbers per unit area were quantified at days 1 and 3 post-wounding. Results are means±s.e.m., n=10 for each time point and group. *P<0.05 compared with wild-type.

FIG. 2: Addition of TGF-β1 to Smad3^{-/-} wounds has no effect on re-epithelialization but enhances matrix production. a, Serum levels of TGF-β1 do not differ significantly between phenotypes; n=8 for each group. b, Expression of TGF-β1 is markedly reduced in Smad3-null and heterozygote wound tissue. Values shown are expressed relative to pooled total messenger RNA levels; n=9 per group. At day 3, no expression was detected in wild-type and null tissue. RNase-protection assays showed a decrease in expression of TGF-β2 and TGF-β3 from days 1-3 post-wounding, with no differences between phenotypes. c, Expression of TGF-β1 was detectable but reduced in day-1 wounds of Smad3-null and heterozygote mice. The type-I receptor was barely detectable in all samples. Values shown are expressed relative to pooled total mRNA levels; n=9 per group.

FIG. 3: Smad3 is required for TGF-P induced monocyte chemotaxis and TGF-β expression. a, Smad3-null monocytes showed a significant decrease in chemotaxis to TGF-β1 compared with wild-type cells but a normal response to the classical chemoattractant fMet-Leu-Phe (fMet). Data shown are the means±s.e.m. of five experiments. *P<0.01 compared with media alone. b, Impaired upregulation of TGF-β1 expression by TGF-β itself in Smad3-null monocytes. Data shown are the means±s.e.m. of four experiments. *P<0.01 compared with media alone. Values shown are expressed relative to total mRNA levels. WT+, HT+ and Null+ indicate cells treated with TGF-β for 24 h. c, Expression of integrin α₅ integrin is upregulated by TGF-β treatment in monocytes of all genotypes. *P<0.05. Values are expressed relative to levels of mRNA expressed from the housekeeping gene HPRT.

FIG. 4: Smad3 deletion modulates keratinocyte proliferation and migration. a, TGF-β1 regulates its own expression in keratinocytes; this response is absent in Smad3-null cells. n=20 animals in each group. C, control medium. *P<0.05, treatment versus control. b, TGF-β1 inhibits growth of wild-type and heterozygote keratinocytes, with a partial response in Smad3-null cells. [³H]Tdr, tritiated thymidine. c, Migration of Smad3-null keratinocytes to TGF-β1 and KGF was significantly reduced compared with wild-type cells; **P<0.01, wild-type versus Smad3-null mutants and heterozygotes; *P<0.01, wild-type versus Smad3-null cells. The response of null cells to conditioned medium (CM) was the same as that of the wild-type cells. d, The expression of integrin as in response to TGF-β1 was impaired in null keratinocytes, with maintained upregulation of integrin β₁. *P<0.01, treated versus untreated cells. Syndecan-1 and E-cadherin were weakly expressed in all samples, with no significant differences observed between phenotypes or treatments.

### DETAILED DESCRIPTION OF THE PREFERRED EMBODIMENT

Smad3 is a member of the Smad family of cytoplasmic proteins that functions to mediate signals from TGF-β and activin receptors to promoters of target genes in the nucleus. To identify selective pathways downstream of the TGF-β receptors, we have characterized mice in which the Smad3 gene has been disrupted by homologous recombination. Studies in these mice and sibling wild-type mice showed that the loss of Smad3 is beneficial to normal wound healing. The data implicate Smad3 in vivo both in the inhibition of re-epithelialization, with specific effects on keratinocyte proliferation, and in TGF-β-mediated chemotaxis of both monocytes and keratinocytes. Our results demonstrate that Smad3 mediates in vivo signalling pathways that are inhibitory to wound healing, as its deletion leads to enhanced re-epithelialization and contracted wound areas. The data indicate that the disruption of the Smad3 pathway in vivo, optionally coupled with exogenous TGF signalling through intact alternative pathways, is to be of therapeutic benefit in accelerating all aspects of impaired wound healing.

Additionally, we describe Smad3 inhibitors that can be used as anti-fibrotic agents, which have a protective effect against induction of fibrosis. The data indicate that Smad3 null mice are protected from fibrosis in response to high dose radiation. Inhibitors of Smad3 can be used to prevent fibrosis, including radiation-induced fibrosis.

Definitions

The term “isolated” requires that a material be removed from its original environment (e.g., the natural environment if it is naturally occurring). For example, a naturally occurring polynucleotide or polypeptide present in a living cell is not isolated, but the same polynucleotide or polypeptide, separated from some or all of the coexisting materials in the natural system, is isolated.

The term “purified” does not require absolute purity; rather it is intended as a relative definition, with reference to the purity of the material in its natural state. Purification of natural material to at least one order of magnitude, preferably two or three magnitudes, and more preferably four or five orders of magnitude is expressly contemplated.

The term “enriched” means that the concentration of the material is at least about 2, 5, 10, 100, or 1000 times its natural concentration (for example), advantageously 0.01% by weight. Enriched preparations of about 0.5%, 1%, 5%, 10%, and 20% by weight are also contemplated.

The Smad3 Gene

To date, nine vertebrate Smads have been identified, and these have been divided into subgroups based on their functional role in various pathways. Smad1, 5, and Smad8, all mediate signal transduction from BMPs, while Smad2 and Smad3 mediate signal transduction from TGF-ps and activins. Collectively, these Smads are known as the pathway-restricted Smads and can form homo or heterodimers. Smad4 has been shown to be a shared hetero-oligomerization partner to the pathway-restricted Smads and is known as the common mediator. The last two members of the family, Smad6 and 7, act to inhibit the Smad signaling cascades often by forming unproductive dimers with other Smads and are therefore classified as antagonistic Smads (Heldin et al., Nature, 1997, 390, 465-471; Kretzschmar and Massague, Curr. Opin. Genet. Dev., 1998, 8, 103-111).

The published cDNA sequence of human Smad3 is available as GenBank accession number U68019, herein expressly incorporated by reference in its entirety. (SEQ ID NO:1). The deduced amino acid sequence is provided in SEQ ID NO:2. The genomic sequence is also known.

The Smad3 nucleotide sequences of the invention include: (a) the cDNA sequence given in SEQ ID NO:1; (b) the nucleotide sequence that encodes the amino acid sequence given in SEQ ID NO:2; (c) any nucleotide sequence that hybridizes to the complement of the cDNA sequence given in SEQ ID NO:1 under highly stringent conditions, e.g., hybridization to filter-bound DNA in 0.5 M NaHPO4, 7% sodium dodecyl sulfate (SDS), 1 mM EDTA at 65° C., and washing in 0.1.times. SSC/0.1% SDS at 68° C. (e.g., see Ausubel F. M. et al., eds., 1989, Current Protocols in Molecular Biology, Vol. I, Green Publishing Associates, Inc., and John Wiley & sons, Inc., New York, at p. 2.10.3) and encodes a functionally equivalent gene product; and (d) any nucleotide sequence that hybridizes to the complement of the cDNA sequence given in SEQ ID NO:1 under less stringent conditions, such as moderately stringent conditions, e.g., washing in 0.2.times. SSC/0.1% SDS at 42° C. (Ausubel et al., 1989, supra), yet which still encodes a functionally equivalent gene product. Functional equivalents of Smad3 include naturally occurring Smad3 present in other species, and mutant Smad3s whether naturally occurring or engineered. Aspects of the invention also include degenerate variants of sequences (a) through (d).

Embodiments of the invention also include nucleic acid molecules, preferably DNA molecules, that hybridize to, and are therefore the complements of, the nucleotide sequences (a) through (d), in the preceding paragraph. Such hybridization conditions may be highly stringent or less highly stringent, as described above. In instances wherein the nucleic acid molecules are deoxyoligonucleotides (“oligos”), highly stringent conditions may refer, e.g., to washing in 6×SSC/0.05% sodium pyrophosphate at 37° C. (for 14-base oligos), 48° C. (for 17-base oligos), 55° C. (for 20-base oligos), and 60° C. (for 23-base oligos). These nucleic acid molecules may encode or act as Smad3 antisense molecules, useful, for example, in Smad3 gene regulation (for and/or as antisense primers in amplification reactions of Smad3 gene nucleic acid sequences). With respect to Smad3 gene regulation, such techniques can be used to regulate, for example, radiation-induced fibrosis and/or cutaneous wound healing. Further, such sequences can be used as part of ribozyme and/or triple helix sequences, also useful for Smad3 gene regulation.

In addition to the Smad3 nucleotide sequences described above, full length Smad3 cDNA or gene sequences present in the same species and/or homologs of the Smad3 gene present in other species can be identified and readily isolated, without undue experimentation, by molecular biological techniques well known in the art. The identification of homologs of Smad3 in related species can be useful for developing animal model systems more closely related to humans for purposes of drug discovery. For example, expression libraries of cDNAs synthesized from mRNA derived from the organism of interest can be screened using labeled TGF-β or activin receptors (or Smads involved in forming dimers with Smad3) derived from that species. Alternatively, such cDNA libraries, or genomic DNA libraries derived from the organism of interest can be screened by hybridization using the nucleotides described herein as hybridization or amplification probes. Furthermore, genes at other genetic loci within the genome that encode proteins, which have extensive homology to one or more domains of the Smad3 gene product, can also be identified via similar techniques. In the case of cDNA libraries, such screening techniques can identify clones derived from alternatively spliced transcripts in the same or different species.

Screening can be by filter hybridization, using duplicate filters. The labeled probe can contain at least 15-30 base pairs of the Smad3 cDNA sequence. The hybridization washing conditions used should be of a lower stringency when the cDNA library is derived from an organism different from the type of organism from which the labeled sequence was derived. With respect to the cloning of a human Smad3 homolog, using murine Smad3 probes, for example, hybridization can, for example, be performed at 65° C. overnight in Church's buffer (7% SDS, 250 mM NaHPO4, 2 μM EDTA, 1% BSA). Washes can be done with 2×SSC, 0.1% SDS at 65° C. and then at 0.1×SSC, 0.1% SDS at 65° C.

Low stringency conditions are well known to those of skill in the art, and will vary predictably depending on the specific organisms from which the library and the labeled sequences are derived. For guidance regarding such conditions see, for example, Sambrook et al., 1989, Molecular Cloning, A Laboratory Manual, Cold Springs Harbor Press, N.Y.; and Ausubel et al., 1989, Current Protocols in Molecular Biology, Green Publishing Associates and Wiley Interscience, N.Y.

Alternatively, the labeled Smad3 nucleotide probe can be used to screen a genomic library derived from the organism of interest, again, using appropriately stringent conditions. The identification and characterization of human genomic clones is helpful for designing clinical protocols for protecting against fibrosis and improving wound healing in human patients. For example, sequences derived from regions adjacent to the intron/exon boundaries of the human gene can be used to design primers for use in amplification assays to detect mutations within the exons, introns, splice sites (e.g. splice acceptor and/or donor sites), etc.

Further, a Smad3 gene homolog may be isolated from nucleic acid of the organism of interest by performing PCR using two degenerate oligonucleotide primer pools designed on the basis of amino acid sequences within the Smad3 gene product disclosed herein. The template for the reaction may be cDNA obtained by reverse transcription of mRNA prepared from, for example, human or non-human cell lines or tissue known or suspected to express a Smad3 gene allele.

The PCR product may be subcloned and sequenced to ensure that the amplified sequences represent the sequences of a Smad3 gene. The PCR fragment may then be used to isolate a full length cDNA clone by a variety of methods. For example, the amplified fragment may be labeled and used to screen a cDNA library, such as a bacteriophage cDNA library. Alternatively, the labeled fragment can be used to isolate genomic clones via the screening of a genomic library.

PCR technology may also be utilized to isolate full length cDNA sequences. For example, RNA can be isolated, following standard procedures, from an appropriate cellular or tissue source (i.e., one known, or suspected, to express the Smad3 gene). A reverse transcription reaction may be performed on the RNA using an oligonucleotide primer specific for the most 5′ end of the amplified fragment for the priming of first strand synthesis. The resulting RNA/DNA hybrid may then be “tailed” with guanines using a standard terminal transferase reaction, the hybrid may be digested with RNAase H, and second strand synthesis may then be primed with a poly-C primer. Accordingly, cDNA sequences upstream of the amplified fragment can be isolated. For a review of cloning strategies that may be used, see e.g., Sambrook et al., 1989, supra.

The Smad3 gene sequences can additionally be used to isolate mutant Smad3 gene alleles. Such mutant alleles can be isolated from individuals either known or proposed to have a genotype that contributes to fibrosis and or wound healing. Mutant alleles and mutant allele products can then be utilized in the therapeutic systems described below. Additionally, such Smad3 gene sequences can be used to detect Smad3 gene regulatory (e.g., promoter or promotor/enhancer) defects that can affect fibrosis or wound healing.

A cDNA of a mutant Smad3 gene can be isolated, for example, by using PCR. In this case, the first cDNA strand can be synthesized by hybridizing an oligo-dT oligonucleotide to mRNA isolated from tissue known or suspected to be expressed in an individual putatively carrying the mutant Smad3 allele, and by extending the new strand with reverse transcriptase. The second strand of the cDNA is then synthesized using an oligonucleotide that hybridizes specifically to the 5′ end of the normal gene. Using these two primers, the product is then amplified via PCR, cloned into a suitable vector, and subjected to DNA sequence analysis through methods well known to those of skill in the art. By comparing the DNA sequence of the mutant Smad3 allele to that of the normal Smad3 allele, the mutation(s) responsible for the loss or alteration of function of the mutant Smad3 gene product can be ascertained.

Alternatively, a genomic library can be constructed using DNA obtained from an individual suspected of or known to carry the mutant Smad3 allele, or a cDNA library can be constructed using RNA from a tissue known, or suspected, to express the mutant Smad3 allele. The normal Smad3 gene or any suitable fragment thereof may then be labeled and used as a probe to identify the corresponding mutant Smad3 allele in such libraries. Clones containing the mutant Smad3 gene sequences can then be purified and subjected to sequence analysis according to methods well known to those of skill in the art.

Additionally, an expression library can be constructed utilizing cDNA synthesized from, for example, RNA isolated from a tissue known, or suspected, to express a mutant Smad3 allele in an individual suspected of or known to carry such a mutant allele. In this manner, gene products made by the putatively mutant tissue can be expressed and screened using standard antibody screening techniques in conjunction with antibodies raised against the normal Smad3 gene product, as described, below, in the sections. (For screening techniques, see, for example, Harlow, E. and Lane, eds., 1988, “Antibodies: A Laboratory Manual”, Cold Spring Harbor Press, Cold Spring Harbor.) Additionally, screening can be accomplished by screening with labeled Smad3 fusion proteins. In cases where a Smad3 mutation results in an expressed gene product with altered function (e.g., as a result of a missense or a frameshift mutation), a polyclonal set of antibodies to Smad3 are likely to cross-react with the mutant Smad3 gene product. Library clones detected via their reaction with such labeled antibodies can be purified and subjected to sequence analysis according to methods well known to those of skill in the art.

Aspects of the invention also concern nucleotide sequences that encode mutant Smad3s, peptide fragments of Smad3, truncated Smad3s, and Smad3 fusion proteins. These include, but are not limited to, nucleotide sequences encoding mutant Smad3s described in subsequent sections or peptides corresponding to a domain of Smad3 or portions of these domains; truncated Smad3s in which one or two of the domains is deleted, or a truncated, nonfunctional Smad3 lacking all or a portion of a domain. Nucleotides encoding fusion proteins may include, but are not limited to, full length Smad3, truncated Smad3 or peptide fragments of Smad3 fused to an unrelated protein or peptide, such as for example, a transmembrane sequence, which anchors the Smad3 to the cell membrane; an Ig Fc domain which increases the stability and half life of the resulting fusion protein in the bloodstream; or an enzyme, fluorescent protein, luminescent protein which can be used as a marker.

Embodiments of the invention also concern (a) DNA vectors that contain any of the foregoing Smad3 coding sequences and/or their complements (i.e., antisense); (b) DNA expression vectors that contain any of the foregoing Smad3 coding sequences operatively associated with a regulatory element that directs the expression of the coding sequences; and (c) genetically engineered host cells that contain any of the foregoing Smad3 coding sequences operatively associated with a regulatory element that directs the expression of the coding sequences in the host cell. As used herein, regulatory elements include, but are not limited to, inducible and non-inducible promoters, enhancers, operators and other elements known to those skilled in the art that drive and regulate expression. Such regulatory elements include, but are not limited to, the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeastα-mating factors.

Particular polynucleotides are DNA sequences having three sequential nucleotides, four sequential nucleotides, five sequential nucleotides, six sequential nucleotides, seven sequential nucleotides, eight sequential nucleotides, nine sequential nucleotides, ten sequential nucleotides, eleven sequential nucleotides, twelve sequential nucleotides, thirteen sequential nucleotides, fourteen sequential nucleotides, fifteen sequential nucleotides, sixteen sequential nucleotides, seventeen sequential nucleotides, eighteen sequential nucleotides, nineteen sequential nucleotides, twenty sequential nucleotides, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty,-six, forty-seven, forty-eight, forty-nine, fifty, fifty-one, fifty-two, fifty-three, fifty-four, fifty-five, fifty-six, fifty-seven, fifty-eight, fifty-nine, sixty, sixty-one, sixty-two, sixty-three, sixty-four, sixty-five, sixty-six, sixty-seven, sixty-eight, sixty-nine, seventy, seventy-one, seventy-two, seventy-three, seventy-four, seventy-five, seventy-six, seventy-seven, seventy-eight, seventy-nine, eighty, ninety, one-hundred, two-hundred, or three-hundred or more sequential nucleotides.

Smad3 Proteins and Polypeptides

Smad3 protein, polypeptides and peptide fragments, mutated, truncated or deleted forms of Smad3 and/or Smad3 fusion proteins can be prepared for a variety of uses, including but not limited to, the generation of antibodies, as reagents for research purposes, or the identification of other cellular gene products involved in the regulation of fibrosis and wound healing, as reagents in assays for screening for compounds that can be used in the prevention of fibrosis and improvement of wound healing, and as pharmaceutical reagents useful in protecting against fibrosis and improving wound healing related to Smad3.

The Smad3 amino acid sequences of the invention include the amino acid sequence, or the amino acid sequence encoded by the cDNA or encoded by the gene. Further, Smad3 of other species are encompassed by the invention. In fact, any Smad3 encoded by the Smad3 nucleotide sequences described in the sections above are within the scope of the invention.

Aspects of the invention also encompass proteins that are functionally equivalent to Smad3 encoded by the nucleotide sequences described in the above sections, as judged by any of a number of criteria, including but not limited to, the ability to bind TGF-β or activin receptors or Smads involved in forming dimers with Smad3, the binding affinity for these ligands, the resulting biological effect of Smad3 binding, e.g., signal transduction, a change in cellular metabolism or change in phenotype when the Smad3 equivalent is present in an appropriate cell type, or the regulation of fibrosis or wound healing. Such functionally equivalent Smad3 proteins include, but are not limited to, additions or substitutions of amino acid residues within the amino acid sequence encoded by the Smad3 nucleotide sequences described in the sections above, but which result in a silent change, thus producing a functionally equivalent gene product. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. While random mutations can be made to Smad3 DNA (using random mutagenesis techniques well known to those skilled in the art) and the resulting mutant Smad3s tested for activity, site-directed mutations of the Smad3 coding sequence can be engineered (using site-directed mutagenesis techniques well known to those skilled in the art) to generate mutant Smad3s with altered function, e.g., different binding affinity for TGF-β or activin receptors or Smads involved in forming dimers with Smad3, and/or different signalling capacity.

For example, identical amino acid residues of a mouse form of Smad3 and the human Smad3 homolog can be aligned so that regions of identity are maintained, whereas the variable residues are altered, e.g., by deletion or insertion of an amino acid residue(s) or by substitution of one or more different amino acid residues. Conservative alterations at the variable positions can be engineered in order to produce a mutant Smad3 that retains function; e.g., ligand binding affinity or signal transduction capability or both. Non-conservative changes can be engineered at these variable positions to alter function, e.g., ligand binding affinity or signal transduction capability, or both. Alternatively, where alteration of function is desired, deletion or non-conservative alterations of the conserved regions (i.e., identical amino acids) can be engineered. For example, deletion or non-conservative alterations (substitutions or insertions) of a domain can be engineered to produce a mutant Smad3 that binds a ligand but is signalling-incompetent. Non-conservative alterations to residues of identical amino acids can be engineered to produce mutant Smad3s with altered binding affinity for ligands. The same mutation strategy can also be used to design mutant Smad3s based on the alignment of other non-human Smad3s and the human Smad3 homolog by aligning identical amino acid residues.

Other mutations to the Smad3 coding sequence can be made to generate Smad3s that are better suited for expression, scale up, etc. in the host cells chosen. For example, cysteine residues can be deleted or substituted with another amino acid in order to eliminate disulfide bridges; N-linked glycosylation sites can be altered or eliminated to achieve, for example, expression of a homogeneous product that is more easily recovered and purified from yeast hosts which are known to hyperglycosylate N-linked sites.

Peptides corresponding to one or more domains of Smad3, as well as fusion proteins in which the full length Smad3, a Smad3 peptide or truncated Smad3 is fused to an unrelated protein, are also within the scope of the invention and can be designed on the basis of the Smad3 nucleotide and Smad3 amino acid sequences given in SEQ ID NOS:1 and 2. Such fusion proteins include but are not limited to IgFc fusions which stabilize the Smad3 protein or peptide and prolong half-life in vivo; or fusions to any amino acid sequence that allows the fusion protein to be anchored to the cell membrane; or fusions to an enzyme, fluorescent protein, or luminescent protein which provide a marker function.

While the Smad3 polypeptides and peptides can be chemically synthesized (e.g., see Creighton, 1983, Proteins: Structures and Molecular Principles, W. H. Freeman & Co., N.Y.), large polypeptides derived from Smad3 and the full length Smad3 itself may advantageously be produced by recombinant DNA technology using techniques well known in the art for expressing nucleic acid containing Smad3 gene sequences and/or coding sequences. Such methods can be used to construct expression vectors containing the Smad3 nucleotide sequences and appropriate transcriptional and translational control signals. These methods include, for example, in vitro recombinant DNA techniques, synthetic techniques, and in vivo genetic recombination. See, for example, the techniques described in Sambrook et al., 1989, supra, and Ausubel et al., 1989, supra. Alternatively, RNA capable of encoding Smad3 nucleotide sequences may be chemically synthesized using, for example, synthesizers. See, for example, the techniques described in “Oligonucleotide Synthesis”, 1984, Gait, M. J. ed., IRL Press, Oxford.

A variety of host-expression vector systems can be utilized to express the Smad3 nucleotide sequences described herein. Where the Smad3 peptide or polypeptide is soluble, the peptide or polypeptide can be recovered from the culture,e.g., from the host cell in cases where the Smad3 peptide or polypeptide is not secreted, and from the culture media in cases where the Smad3 peptide or polypeptide is secreted by the cells. However, the expression systems also encompass engineered host cells that express the Smad3 or functional equivalents in situ, e.g., anchored in the cell membrane. Purification or enrichment of the Smad3 from such expression systems can be accomplished using appropriate detergents and lipid micelles and methods well known to those skilled in the art. However, such engineered host cells themselves may be used in appropriate situations.

The expression systems that may be used with some embodiments include, but are not limited to, microorganisms such as bacteria (e.g., E. coli, B. subtilis) transformed with recombinant bacteriophage DNA, plasmid DNA or cosmid DNA expression vectors containing Smad3 nucleotide sequences; yeast (e.g., Saccharomyces, Pichia) transformed with recombinant yeast expression vectors containing the Smad3 nucleotide sequences; insect cell systems infected with recombinant virus expression vectors (e.g., baculovirus) containing the Smad3 sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing Smad3 nucleotide sequences; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the Smad3 gene product being expressed. For example, when a large quantity of such a protein is to be produced, for the generation of pharmaceutical compositions of Smad3 protein or for raising antibodies to the Smad3 protein, for example, vectors which direct the expression of high levels of fusion protein products that are readily purified may be desirable. Such vectors include, but are not limited, to the E. coli expression vector pUR278 (Ruther et al., 1983, EMBO J. 2:1791), in which the Smad3 coding sequence may be ligated individually into the vector in frame with the lacZ coding region so that a fusion protein is produced; pIN vectors (Inouye & Inouye, 1985, Nucleic Acids Res. 13:3101-3109; Van Heeke & Schuster, 1989, J. Biol. Chem. 264:5503-5509); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The PGEX vectors are designed to include thrombin or factor Xa protease cleavage sites so that the cloned target gene product can be released from the GST moiety.

In an insect system, Autographa californica nuclear polyhidrosis virus (AcNPV) is used as a vector to express foreign genes. The virus grows in Spodoptera frugiperda cells. The Smad3 gene coding sequence may be cloned individually into non-essential regions (for example the polyhedrin gene) of the virus and placed under control of an AcNPV promoter (for example the polyhedrin promoter). Successful insertion of Smad3 gene coding sequence will result in inactivation of the polyhedrin gene and production of non-occluded recombinant virus, (i.e., virus lacking the proteinaceous coat coded for by the polyhedrin gene). These recombinant viruses are then used to infect Spodoptera frugiperda cells in which the inserted gene is expressed. (E.g., see Smith et al., 1983, J. Virol. 46: 584; Smith, U.S. Pat. No. 4,215,051).

In mammalian host cells, a number of viral-based expression systems may be utilized. In cases where an adenovirus is used as an expression vector, the Smad3 nucleotide sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the Smad3 gene product in infected hosts. (E.g., See Logan & Shenk, 1984, Proc. Natl. Acad. Sci. USA 81:3655-3659). Specific initiation signals may also be required for efficient translation of inserted Smad3 nucleotide sequences. These signals include the ATG initiation codon and adjacent sequences. In cases where an entire Smad3 gene or cDNA, including its own initiation codon and adjacent sequences, is inserted into the appropriate expression vector, no additional translational control signals may be needed. However, in cases where only a portion of the Smad3 coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (See Bittner et al., 1987, Methods in Enzymol. 153:516-544).

In addition, a host cell strain that modulates the expression of the inserted sequences, or modifies and processes the gene product in the specific fashion desired may be chosen. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, and WI38.

For long-term, high-yield production of recombinant proteins, stable expression is preferred. For example, cell lines which stably express the Smad3 sequences described above may be engineered. Rather than using expression vectors which contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the Smad3 gene product. Such engineered cell lines may be particularly useful in screening and evaluation of compounds that affect the endogenous activity of the Smad3 gene product.

A number of selection systems may be used, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes can be employed in tk-, hgprt- or aprt-cells, respectively. Also, antimetabolite resistance can be used as the basis of selection for the following genes: dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147).

Alternatively, any fusion protein may be readily purified by utilizing an antibody specific for the fusion protein being expressed. For example, a system described by Janknecht et al. allows for the ready purification of non-denatured fusion proteins expressed in human cell lines (Janknecht, et al., 1991, Proc. Natl. Acad. Sci. USA 88: 8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺.nitriloacetic acid-agarose columns and histidine-tagged proteins are selectively eluted with imidazole-containing buffers.

The Smad3 gene products can also be expressed in transgenic animals. Animals of any species, including, but not limited to, mice, rats, rabbits, guinea pigs, pigs, micro-pigs, goats, and non-human primates, e.g., baboons, monkeys, and chimpanzees may be used to generate Smad3 transgenic animals.

Particular polypeptides are amino acid sequences having three sequential residues, four sequential residues, five sequential residues, six sequential residues, seven sequential residues, eight sequential residues, nine sequential residues, ten sequential residues, eleven sequential residues, twelve sequential residues, thirteen sequential residues, fourteen sequential residues, fifteen sequential residues, sixteen sequential residues, seventeen sequential residues, eighteen sequential residues, nineteen sequential residues, twenty sequential residues, twenty-one, twenty-two, twenty-three, twenty-four, twenty-five, twenty-six, twenty-seven, thirty, forty, fifty, sixty, seveny, eighty, ninety, or more sequential residues.

Screening Assays for Compounds that Inhibit Smad3 Expression or Activity

The following assays are designed to identify compounds that inhibit Smad3, compounds that interfere with the interaction of Smad3 with intracellular proteins, and compounds that interfere with the interaction of Smad3 with transmembrane proteins, e.g., TGF-β and activin receptors, and compounds, which inhibit the activity of the Smad3 gene or modulate the level of Smad3. Assays may additionally be utilized which identify compounds which bind to Smad3 gene regulatory sequences (e.g., promoter sequences) and which may inhibit Smad3 gene expression. Assays may additionally be utilized to identify compounds which interfere with the interaction of Smad3 with promoters of target genes.

The compounds that may be screened in accordance with these embodiments include, but are not limited to: peptides, antibodies and fragments thereof, and other organic compounds (e.g., peptidomimetics) that bind to Smad3, or to intracellular proteins that interact with Smad 3, or to transmembrane proteins that interact with Smad3 and inhibit the activity triggered by Smad3 or mimic the inhibitors of Smad3; as well as peptides, antibodies or fragments thereof, and other organic compounds that mimic the ligands of Smad3 (or a portion thereof) and bind to and “neutralize” Smad3.

Such compounds may include, but are not limited to, peptides such as, for example, soluble peptides, including but not limited to members of random peptide libraries; (see, e.g., Lam, K. S. et al., 1991, Nature 354:82-84; Houghten, R. et al., 1991, Nature 354:84-86), and combinatorial chemistry-derived molecular libraries made of D- and/or L-configuration amino acids, phosphopeptides (including, but not limited to, members of random or partially degenerate, directed phosphopeptide libraries; see, e.g., Songyang, Z. et al., 1993, Cell 72:767-778), antibodies (including, but not limited to, polyclonal, monoclonal, humanized, anti-idiotypic, chimeric or single chain antibodies, and FAb, F(ab′)2 and FAb expression library fragments, and epitope-binding fragments thereof), and small organic or inorganic molecules.

Other compounds that can be screened in accordance with these embodiments include but are not limited to small organic molecules that affect the expression of the Smad3 gene or some other gene balancing the interaction of intracellular proteins with Smad3 or the interaction of transmembrane proteins with Smad3 (e.g., by interacting with the regulatory region or transcription factors involved in gene expression); or such compounds that affect the activity of Smad3 or the activity of some other intracellular protein that interacts with Smad3 or of some other transmembrane protein that interacts with Smad3 or of promoters of target genes regulated by Smad3.

Computer modelling and searching technologies permit identification of compounds, or the improvement of already identified compounds, that can inhibit Smad3 expression or activity. Having identified such a compound or composition, the active sites or regions are identified. Such active sites might typically be ligand binding sites, such as the interaction domains of the ligand with Smad3 itself. The active site can be identified using methods known in the art including, for example, from the amino acid sequences of peptides, from the nucleotide sequences of nucleic acids, or from study of complexes of the relevant compound or composition with its ligand. In the latter case, chemical or X-ray crystallographic methods can be used to find the active site by finding where on the factor the complexed ligand is found. Next, the three dimensional geometric structure of the active site is determined. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric structures. The geometric structures may be measured with a complexed ligand, natural or artificial, which may increase the accuracy of the active site structure determined. Indeed, the Smad interaction domains have been determined for known inhibitors of Smad3, including the transcriptional repressors TGIF and SIP1, the adenoviral oncoprotein E1A, and the human oncogenes Ski, SnoN, and Evi-1 and may serve as the basis for rational drug design.

If an incomplete or insufficiently accurate structure is determined, the methods of computer based numerical modeling can be used to complete the structure or improve its accuracy. Any recognized modeling method can be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site, either experimentally, by modeling, or by a combination, candidate inhibiting compounds can be identified by searching databases containing compounds along with information on their molecular structure. Such a search seeks compounds having structures that match the determined active site structure and that interact with the groups defining the active site. Such a search can be manual, but is preferably computer assisted. The compounds found from this search are potential Smad3 inhibiting compounds.

Alternatively, these methods can be used to identify improved inhibiting compounds from an already known inhibiting compound or ligand. The composition of the known compound can be modified and the structural effects of modification can be determined using the experimental and computer modeling methods described above applied to the new composition. The altered structure is then compared to the active site structure of the compound to determine if an improved fit or interaction results. In this manner systematic variations in composition, such as by varying side groups, can be quickly evaluated to obtain modified inhibiting compounds or ligands of improved specificity or activity.

Further experimental and computer modeling methods useful to identify inhibiting compounds will be apparent to those of skill in the art based upon identification of the active sites of Smad3, and of intracellular and transmembrane proteins that interact with Smad3, and of related transduction and transcription factors, as well as of promoters of target genes regulated by Smad3.

Examples of molecular modelling systems are the CHARMM and QUANTA programs (Polygen Corporation, Waltham, Mass.). CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific-proteins, such as Rotivinen, et al., 1988, Acta Pharmaceutical Fennica 97:159-166; Ripka, New Scientist 54-57 (Jun. 16, 1988); McKinaly and Rossmann, 1989, Annu. Rev. Pharmacol. Toxiciol. 29:111-122; Perry and Davies, OSAR: Quantitative Structure-Activity Relationships in Drug Design pp. 189-193 (Alan R. Liss, Inc. 1989); Lewis and Dean, 1989 Proc. R. Soc. Lond. 236:125-140 and 141-162; and, with respect to a model receptor for nucleic acid components, Askew, et al., 1989, J. Am. Chem. Soc. 111:1082-1090. Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario). Although these are primarily designed for application to drugs specific to particular proteins, they can be adapted to design of drugs specific to regions of DNA or RNA, once that region is identified.

Although described above with reference to design and generation of compounds that could alter binding, one could also screen libraries of known compounds, including natural products or synthetic chemicals, and biologically active materials, including proteins, for compounds which are inhibitors of Smad3.

Compounds identified via assays such as those described herein may be useful, for example, in elaborating the biological function of the Smad3 gene product, and for preventing fibrosis and improving wound healing.

In vitro Screening Assays for Compounds that Bind to Smad3

In vitro systems can be designed to identify compounds capable of interacting with (e.g., binding to) Smad3. Compounds identified are useful, for example, in inhibiting the activity of wild type and/or mutant Smad3 gene products; are useful in elaborating the biological function of Smad3; can be utilized in screens for identifying compounds that disrupt normal Smad3 interactions; or can in themselves disrupt such interactions.

The principle of the assays used to identify compounds that bind to Smad3 involves preparing a reaction mixture of Smad3 and the test compound under conditions and for a time sufficient to allow the two components to interact and bind, thus forming a complex which can be removed and/or detected in the reaction mixture. The Smad3 species used can vary depending upon the goal of the screening assay. For example, where compounds that bind and inhibit or mimic the inhibitors or mimic the ligands of Smad3 and bind to and “neutralize” Smad3 are sought, the full length Smad3 protein, a peptide corresponding to a domain or a fusion protein containing a Smad3 domain fused to a protein or polypeptide that affords advantages in the assay system (e.g., labeling, isolation of the resulting complex, etc.) can be utilized.

The screening assays can be conducted in a variety of ways. For example, one method to conduct such an assay would involve anchoring the Smad3 protein, polypeptide, peptide or fusion protein or the test substance onto a solid phase and detecting Smad3/test compound complexes anchored on the solid phase at the end of the reaction. In one embodiment of such a method, the Smad3 reactant can be anchored onto a solid surface, and the test compound, which is not anchored, can be labeled, either directly or indirectly.

In practice, microtiter plates are conveniently utilized as the solid phase. The anchored component can be immobilized by non-covalent or covalent attachments. Non-covalent attachment can be accomplished by simply coating the solid surface with a solution of the protein and drying. Alternatively, an immobilized antibody, preferably a monoclonal antibody, specific for the protein to be immobilized can be used to anchor the protein to the solid surface. The surfaces can be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the previously non-immobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for Smad3 protein, polypeptide, peptide or fusion protein or the test compound to anchor any complexes formed in solution, and a labeled antibody specific for the other component of the possible complex to detect anchored complexes.

Alternatively, cell-based assays can be used to identify compounds that interact with Smad3. To this end, cell lines that express Smad3, or cell lines (e.g., COS cells, CHO cells, fibroblasts, etc.) that have been genetically engineered to express Smad3 (e.g., by transfection or transduction of Smad3 DNA) can be used. Interaction of the test compound with, for example, the Smad3 expressed by the host cell can be determined by comparison or competition with native ligand.

Assays for Intracellular or Transmembrane Proteins that Interact with the Smad3

Any method suitable for detecting protein-protein interactions may be employed for identifying transmembrane proteins or intracellular proteins that interact with Smad3. Among the traditional methods which may be employed are co-immunoprecipitation, crosslinking and co-purification through gradients or chromatographic columns of cell lysates or proteins obtained from cell lysates and the Smad3 protein to identify proteins in the lysate that interact with the Smad3 protein. For these assays, the Smad3 component used can be a full length Smad3 protein, a peptide corresponding to a domain of Smad3 or a fusion protein containing a domain of Smad3. Once isolated, such an intracellular or transmembrane protein can be identified and can, in turn, be used, in conjunction with standard techniques, to identify proteins with which it interacts. For example, at least a portion of the amino acid sequence of an intracellular or transmembrane protein which interacts with Smad3 can be ascertained using techniques well known to those of skill in the art, such as via the Edman degradation technique. (See, e.g., Creighton, 1983, “Proteins: Structures and Molecular Principles”, W. H. Freeman & Co., N.Y., pp.34-49). The amino acid sequence obtained can be used as a guide for the generation of oligonucleotide mixtures that can be used to screen for gene sequences encoding such intracellular and transmembrane proteins. Screening can be accomplished, for example, by standard hybridization or PCR techniques. Techniques for the generation of oligonucleotide mixtures and the screening are well-known. (See, e.g., Ausubel et al., 1989, Current Protocols in Molecular Biology, Green Publishing Associates and Wiley Interscience, N.Y., and PCR Protocols: A Guide to Methods and Applications, 1990, Innis, M. et al., eds. Academic Press, Inc., New York).

Additionally, methods can be employed that result in the simultaneous identification of genes, which encode the transmembrane or intracellular proteins interacting with Smad3. These methods include, for example, probing expression, libraries, in a manner similar to the well known technique of antibody probing of λgt11 libraries, using labeled Smad3 protein, or a Smad3 polypeptide, peptide or fusion protein, e.g., a Smad3 polypeptide or Smad3 domain fused to a marker (e.g., an enzyme, fluor, luminescent protein, or dye), or an Ig-Fc domain.

One method which detects protein interactions in vivo, the two-hybrid system, is described in detail for illustration only and not by way of limitation. One version of this system has been described (Chien et al., 1991, Proc. Natl. Acad. Sci. USA, 88:9578-9582) and is commercially available from Clontech (Palo Alto, Calif.). The assay identifies proteins that interact with Smad3, whether physiologically or pharmacologically.

Briefly, utilizing such a system, plasmids are constructed that encode two hybrid proteins: one plasmid consists of nucleotides encoding the DNA-binding domain of a transcription activator protein fused to a Smad3 nucleotide sequence encoding Smad3, a Smad3 polypeptide, peptide or fusion protein, and the other plasmid consists of nucleotides encoding the transcription activator protein's activation domain fused to a cDNA encoding an unknown protein, which has been recombined into this plasmid as part of a cDNA library. The DNA-binding domain fusion plasmid and the cDNA library are transformed into a strain of the yeast Saccharomyces cerevisiae that contains a reporter gene whose regulatory region contains the transcription activator's binding site. Either hybrid protein alone cannot activate transcription of the reporter gene: the DNA-binding domain hybrid cannot because it does not provide activation function and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology may be used to screen activation domain libraries for proteins that interact with the “bait” gene product. By way of example, and not by way of limitation, Smad3 may be used as the bait gene product. Total genomic or cDNA sequences are fused to the DNA encoding an activation domain. This library and a plasmid encoding a hybrid of a bait Smad3 gene product fused to the DNA-binding domain are co-transformed into a yeast reporter strain, and the resulting transformants are screened for those that express the reporter gene. For example, and not by way of limitation, a bait Smad3 gene sequence, such as the open reading frame of Smad3 (or a domain of Smad3), can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with bait Smad3 gene product are to be detected can be made using methods routinely practiced in the art. According to the particular system described herein, for example, the cDNA fragments can be inserted into a vector such that they are translationally fused to the transcriptional activation domain of GAL4. This library can be co-transformed along with the bait Smad3 gene-GAL4 fusion plasmid into a yeast strain which contains a lacZ gene driven by a promoter which contains GAL4 activation sequence. A cDNA encoded protein, fused to GAL4 transcriptional activation domain, that interacts with bait Smad3 gene product will reconstitute an active GAL4 protein and thereby drive expression of the HIS3 gene. Colonies which express HIS3 can be detected by their growth on petri dishes containing semi-solid agar based media lacking histidine. The cDNA can then be purified from these strains, and used to produce and isolate the bait Smad3 gene-interacting protein using techniques routinely practiced in the art.

Assays for Compounds that Interfere with Smad3/Intracellular or Smad3/Transmembrane Macromolecule Interaction

The macromolecules that interact with Smad3 are referred to, for purposes of this discussion, as “ligands”. These ligands are likely to be involved in the Smad3 signal transduction pathway, and therefore, in the role of Smad3 in wound healing and fibrosis. Therefore, it is desirable to identify compounds that interfere with or disrupt the interaction of such ligands with Smad3, which may be useful in regulating the activity of Smad3 and control wound healing and fibrosis associated with Smad3 activity.

The basic principle of the assay systems used to identify compounds that interfere with the interaction between Smad3 and its ligand or ligands involves preparing a reaction mixture containing the Smad3 protein, polypeptide, peptide or fusion protein and the ligand under conditions and for a time sufficient to allow the two to interact and bind, thus forming a complex. In order to test a compound for inhibitory activity, the reaction mixture is prepared in the presence and absence of the test compound. The test compound may be initially included in the reaction mixture, or may be added at a time subsequent to the addition of the Smad3 moiety and its ligand. Control reaction mixtures are incubated without the test compound or with a placebo. The formation of any complexes between the Smad3 moiety and the ligand is then detected. The formation of a complex in the control reaction, but not in the reaction mixture containing the test compound, indicates that the compound interferes with the interaction of Smad3 and the interactive ligand. Additionally, complex formation within reaction mixtures containing the test compound and normal Smad3 protein can also be compared to complex formation within reaction mixtures containing the test compound and a mutant Smad3. This comparison may be important in those cases wherein it is desirable to identify compounds that disrupt interactions of mutant but not normal Smad3 proteins, for example.

The assay for compounds that interfere with the interaction of Smad3 and ligands can be conducted in a heterogeneous or homogeneous format. Heterogeneous assays involve anchoring either the Smad3 moiety product or the ligand onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction by competition can be identified by conducting the reaction in the presence of the test substance; i.e., by adding the test substance to the reaction mixture prior to or simultaneously with the Smad3 moiety and interactive ligand. Alternatively, test compounds that disrupt preformed complexes, e.g. compounds with higher binding constants that displace one of the components from the complex, can be tested by adding the test compound to the reaction mixture after complexes have been formed. The various formats are described briefly below.

In a heterogeneous assay system, either the Smad3 moiety or the interactive ligand, is anchored onto a solid surface, while the non-anchored species is labeled, either directly or indirectly. In practice, microtiter plates are conveniently utilized. The anchored species may be immobilized by non-covalent or covalent attachments. Non-covalent attachment can be accomplished simply by coating the solid surface with a solution of the Smad3 gene product or ligand and drying. Alternatively, an immobilized antibody specific for the species to be anchored may be used to anchor the species to the solid surface. The surfaces can be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, may be directly labeled or indirectly labeled with a labeled anti-Ig antibody). Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds which inhibit complex or which disrupt preformed complexes can be identified.

In an alternate embodiment of the invention, a homogeneous assay can be used. In this approach, a preformed complex of the Smad3 moiety and the interactive ligand is prepared in which either the Smad3 or its ligand is labeled, but the signal generated by the label is quenched due to formation of the complex (see, e.g., U.S. Pat. No. 4,109,496 by Rubenstein, which utilizes this approach for immunoassays). The addition of a test substance that competes with and displaces one of the species from the preformed complex will result in the generation of a signal above background. In this way, test substances which disrupt Smad3/ligand interaction can be identified.

In a particular embodiment, a Smad3 fusion can be prepared for immobilization. For example, Smad3, or a peptide fragment, e.g., corresponding to a domain, can be fused to a glutathione-S-transferase (GST) gene using a fusion vector, such as pGEX-5X-1, in such a manner that its binding activity is maintained in the resulting fusion protein. The interactive ligand can be purified and used to raise a monoclonal antibody, using methods routinely practiced in the art. This antibody can be labeled with the radioactive isotope ¹²⁵I, for example, by methods routinely practiced in the art. In a heterogeneous assay, e.g., the GST-Smad3 fusion protein can be anchored to glutathione-agarose beads. The interactive ligand can then be added in the presence or absence of the test compound in a manner that allows interaction and binding to occur. At the end of the reaction period, unbound material can be washed away, and the labeled monoclonal antibody can be added to the system and allowed to bind to the complexed components. The interaction between the Smad3 gene product and the interactive ligand can be detected by measuring the amount of radioactivity that remains associated with the glutathione-agarose beads. A successful inhibition of the interaction by the test compound will result in a decrease in measured radioactivity.

Alternatively, the GST-Smad3 fusion protein and the interactive ligand can be mixed together in liquid in the absence of the solid glutathione-agarose beads. The test compound can be added either during or after the species are allowed to interact. This mixture can then be added to the glutathione-agarose beads and unbound material is washed away. Again the extent of inhibition of the Smad3/ligand interaction can be detected by adding the labeled antibody and measuring the radioactivity associated with the beads.

In another embodiment of the invention, these same techniques can be employed using peptide fragments that correspond to the binding domains of Smad3 and/or the interactive ligand (in cases where the ligand is a protein), in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex can then be selected. Sequence analysis of the genes encoding the respective proteins will reveal the mutations that correspond to the region of the protein involved in interactive binding. Alternatively, one protein can be anchored to a solid surface using methods described above, and allowed to interact with and bind to its labeled ligand, which has been treated with a proteolytic enzyme, such as trypsin. After washing, a short, labeled peptide comprising the binding domain may remain associated with the solid material, which can be isolated and identified by amino acid sequencing. Also, once the gene coding for the interactive ligand is obtained, short gene segments can be engineered to express peptide fragments of the protein, which can then be tested for binding activity and purified or synthesized.

For example, and not by way of limitation, a Smad3 gene product can be anchored to a solid material as described above, by making a GST-Smad3 fusion protein and allowing it to bind to glutathione agarose beads. The interactive ligand can be labeled with a radioactive isotope, such as ³⁵S, and cleaved with a proteolytic enzyme such as trypsin. Cleavage products can then be added to the anchored GST-Smad3 fusion protein and allowed to bind. After washing away unbound peptides, labeled bound material, representing the interactive ligand binding domain, can be eluted, purified, and analyzed for amino acid sequence by well-known methods. Peptides so identified can be produced synthetically or fused to appropriate facilitative proteins using recombinant DNA technology.

In one embodiment, the “ligand” is Smad4, with which Smad3 heteroligomerizes upon receptor activation. In another embodiment, the “ligand” is SARA (Smad anchor for receptor activation), which recruits the cytoplasmic signal transducer Smad3. In a further embodiment, the “ligand” is the cognate DNA binding site for Smad3. Smad MH2 domains are the locus of Smad-dependent transcriptional activation activity, and are the site of protein-protein interactions responsible for oligomerization of Smad proteins as well as heteromerization with other transcription factors. Thus, in some embodiments, the MH2 domain of Smad3 is substituted for Smad3 itself in the assays described herein.

Assays for Identification of Compounds that Prevent Fibrosis or Improve Wound Healing

Compounds including, but not limited to, binding compounds identified via assay techniques such as those described in the preceding sections, can be tested for the ability to prevent fibrosis and improve wound healing. The assays described above can identify compounds that affect Smad3 activity (e.g., compounds that bind to Smad3, inhibit binding of a natural ligand, and either block activation (antagonists) or mimic inhibitors of activation (agonists), and compounds that bind to a natural ligand of Smad3 and neutralize ligand activity); or compounds that affect Smad3 gene activity (by affecting Smad3 gene expression, including molecules, e.g., proteins or small organic molecules, that affect or interfere with splicing events so that expression of the full length or a truncated form of Smad3 can be modulated). However, it should be noted that the assays described can also identify compounds that inhibit Smad3 signal transduction (e.g., compounds which affect upstream or downstream signalling events). The identification and use of such compounds that affect another step in the Smad3 signal transduction pathway in which the Smad3 gene and/or Smad3 gene product is involved and, by affecting this same pathway may modulate the effect of Smad3 on fibrosis and wound healing are within the scope of the invention. Such compounds can be used as part of a method for the prevention of fibrosis and improvement of wound healing.

Aspects of the invention also encompass cell-based and animal model-based assays for the identification of compounds exhibiting such an ability to prevent fibrosis and improve wound healing.

Cell-based systems can be used to identify compounds that act to prevent fibrosis and improve wound healing. Such cell systems can include, for example, recombinant or non-recombinant cells, such as cell lines, which express the Smad3 gene. For example monocyte cells, keratinocyte cells, or cell lines derived from monocytes or keratinocytes can be used.

In utilizing such cell systems, cells are exposed to a compound suspected of exhibiting an ability to protect against fibrosis and improve wound healing, at a sufficient concentration and for a time sufficient to elicit a cellular phenotype associated with such a protection against fibrosis and improvement of wound healing in the exposed cells, e.g., altered migration and selective chemotactic response to TGF-β. After exposure, the cells can be assayed to measure alterations in the expression of the Smad3 gene, e.g., by assaying cell lysates for Smad3 mRNA transcripts (e.g., by Northern analysis) or for Smad3 protein expressed in the cell; compounds which inhibit expression of the Smad3 gene are good candidates as therapeutics. Alternatively, the cells are examined to determine whether one or more cellular phenotype associated with fibrosis or impaired wound healing has been altered to resemble a cellular phenotype associated with protection against fibrosis and improvement of wound healing. Still further, the expression and/or activity of components of the signal transduction pathway of which Smad3 is a part, or the activity of Smad3 signal transduction pathway itself can be assayed.

For example, after exposure, the cell lysates can be assayed for the presence of host cell proteins, as compared to lysates derived from unexposed control cells. The ability of a test compound to inhibit expression of specific Smad3 target genes in these assay systems indicates that the test compound inhibits signal transduction initiated by Smad3 activation. The cell lysates can be readily assayed using a Western blot format; i.e., the host cell proteins are resolved by gel electrophoresis, transferred and probed using a anti-host cell protein detection antibody (e.g., an anti-host cell protein detection antibody labeled with a signal generating compound, such as radiolabel, fluor, enzyme, etc.). Alternatively, an ELISA format could be used in which a particular host cell protein is immobilized using an antibody specific for the target host cell protein, and the presence or absence of the immobilized host cell protein is detected using a labeled second antibody. In yet another approach, ion flux, such as calcium ion flux, can be measured as an end point for Smad3 stimulated signal transduction. In yet a further approach, assays for compounds that interfere with Smad3 binding to its cognate DNA binding site utilize specific reporter constructs, such as (SBE)4-luciferase reporter, driven by four repeats of the sequence identified as a Smad binding element in the JunB promoter.

In addition, animal-based systems for protection against fibrosis and improvement of wound healing, for example, may be used to identify compounds capable of protecting against fibrosis and improving wound healing. Such animal models may be used as test substrates for the identification of drugs, pharmaceuticals, therapies and interventions which may be effective in protecting against fibrosis and improving wound healing. For example, animal models can be exposed to a compound, suspected of protecting against fibrosis or improving wound healing, at a sufficient concentration and for a time sufficient to elicit a protection against fibrosis and improvement of wound healing in the exposed animals. The response of animals to the exposure can be monitored by assessing radioprotection or cutaneous wound healing. With regard to intervention, any treatments which protect against any aspect of fibrosis or improve any aspect of wound healing should be considered as candidates for human therapeutic intervention in protecting against fibrosis and improving wound healing. Dosages of test agents may be determined by deriving dose-response curves, as discussed in the sections below.

Inhibition of Smad3 Expression or Smad3 Activity to Prevent Fibrosis or Improve Wound Healing

Any method that neutralizes Smad3 or inhibits expression of the Smad3 gene (either transcription or translation) can be used to protect against fibrosis and improve wound healing. Such approaches can be used to reduce the size of wounds, to treat chronic non-healing wounds, to promote closure in surgical wounds, to speed the re-epithelialization of wounds, to treat ulcers, e.g., decubitus ulcers, diabetic ulcers, and venous stasis ulcers, to improve the growth of autologous skin grafts, and to hasten the recovery of severe burn patients. Such methods can also be useful for imparting resistance to fibrosis resulting from chronic inflammation, e.g., pulmonary fibrosis, glomerulosclerosis, and cirrhosis, protecting against radiation-induced fibrosis, supporting members of the armed forces who might be expected to encounter high dose radiation, permitting dose escalation of radiation treatment, e.g., in cancer patients, and decreasing the accumulation of scar tissue.

For example, the administration of soluble peptides, proteins, fusion proteins, or antibodies (including anti-idiotypic antibodies) that bind to and “neutralize” Smad3 can be used to protect against fibrosis and improve wound healing. To this end, peptides corresponding to the cytoplasmic domain of the TGF-β or activin receptor (or a domain of a Smad involved in forming dimers with Smad3) can be utilized. Alternatively, anti-idiotypic antibodies or Fab fragments of antiidiotypic antibodies that mimic the cytoplasmic domain of the TGF-β or activin receptor (or the domain of a Smad involved in forming dimers with Smad3) and that neutralize Smad3 can be used. Such Smad3 peptides, proteins, fusions proteins, antibodies, anti-idiotypic antibodies or Fabs are administered to a subject in amounts sufficient to neutralize Smad3 and protect against fibrosis or improve wound healing.

In some embodiments, the peptides, proteins, fusions proteins, antibodies, anti-idiotypic antibodies or Fabs are cell-permeable compounds. In other embodiments, cells are genetically engineered using recombinant DNA techniques to introduce the coding sequence for the peptide, protein, fusion protein, antibody, anti-idiotypic antibody or Fab into the cell, e.g., by transduction (using viral vectors, such as retroviruses, adenoviruses, and adeno-associated viruses) or transfection procedures, including but not limited to, the use of naked DNA or RNA, plasmids, cosmids, YACs, electroporation, liposomes, etc. The coding sequence can be placed under the control of a strong constitutive or inducible promoter, or a tissue-specific promoter, to achieve expression of the gene product. The engineered cells that express the gene product can be produced in vitro and introduced into the patient, e.g., systemically, intraperitoneally, at the site of cutaneous wound healing, or the cells can be incorporated into a matrix and implanted in the body, e.g., genetically engineered cells can be implanted as part of a skin graft. Alternatively, the engineered cells that express the gene product can be produced following in vivo gene therapy approaches.

In a preferred embodiment, monoclonal antibodies are produced in one of three different ways. They can be generated as mouse antibodies that are subsequently “humanized” by recombination with human antibody genes (Kohler and Milstein, Nature 256,495 (1975); Winter and Harris, Trends Pharmacol. Sci. 14, 139 (1993); and Queen et al., Proc. Natl. Acad. Sci. USA 86, 10029 (1989)). Alternatively, human antibodies are raised in nude mice grafted with human immune cells (Bruggemann and Neuberger, Immunol. Today 8, 391 (1996)). Finally antibodies can also be made by phase display techniques (Huse et al., Science 246, 1275 (1989); Hoogenboom et al., Immunotechnology 4, 1 (1998); and Rodi and Makowski, Curr. Opin. Biotechnol. 10, 87 (1999)).

For the production of antibodies, various host animals may be immunized by injection with Smad3, a Smad3 peptide, functional equivalents or mutants of Smad3. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Polyclonal antibodies are heterogeneous populations of antibody molecules derived from the sera of the immunized animals.

Monoclonal antibodies, which are homogeneous populations of antibodies to a particular antigen, can be obtained by any technique which provides for the production of antibody molecules by continuous cell lines in culture. These include, but are not limited to, the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb can be cultivated in vitro or in vivo. Production of high titers of mAbs in vivo is preferred.

In addition, techniques developed for the production of “chimeric antibodies” (Morrison et al., 1984, Proc. Natl. Acad. Sci., 81:6851-6855; Neuberger et al., 1984, Nature, 312:604-608; Takeda et al., 1985, Nature, 314:452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can be adapted to produce single chain antibodies against Smad3 gene products. Single chain antibodies are formed by linking the heavy and light chain fragments of the Fv region via an amino acid bridge, resulting in a single chain polypeptide.

Antibody fragments which recognize specific epitopes may be generated by known techniques. For example, such fragments include but are not limited to: the F(ab′)2 fragments which can be produced by pepsin digestion of the antibody molecule and the Fab fragments which can be generated by reducing the disulfide bridges of the F(ab′)2 fragments. Alternatively, Fab expression libraries may be constructed (Huse et al., 1989, Science, 246:1275-1281) to allow rapid and easy identification of monoclonal Fab fragments with the desired specificity.

Antibodies to ligands of Smad3 can, in turn, be utilized to generate anti-idiotype antibodies that “mimic” these ligands, using techniques well known to those skilled in the art. (See, e.g., Greenspan & Bona, 1993, FASEB J 7(5):437-444; and Nissinoff, 1991, J. Immunol. 147(8):2429-2438). For example antibodies that bind to the cytoplasmic domain of the TGF-β or activin receptor (or the domain of a Smad involved in forming dimers with Smad3) and competitively inhibit the binding of Smad3 to the TGF-β or activin receptor (or a Smad involved in forming dimers with Smad3) can be used to generate anti-idiotypes that “mimic” these ligands and, therefore, bind and neutralize Smad3. Such neutralizing anti-idiotypes or Fab fragments of such anti-idiotypes can be used in therapeutic regimens to neutralize Smad3 and protect against fibrosis and improve wound healing.

In an alternate embodiment, interventions to prevent fibrosis and improve wound healing can be designed by reducing the level of endogenous Smad3 gene expression, e.g., using antisense or ribozyme approaches to inhibit or prevent translation of Smad3 mRNA transcripts; triple helix approaches to inhibit transcription of the Smad3 gene; or targeted homologous recombination to inactivate or “knock out” the Smad3 gene or its endogenous promoter. Delivery techniques are preferably designed for a systemic approach. Alternatively, the antisense, ribozyme or DNA constructs described herein can be administered directly to the site containing the target cells, e.g., sites of cutaneous wound healing.

Antisense approaches involve the design of oligonucleotides (either DNA or RNA) that are complementary to Smad3 mRNA. The antisense oligonucleotides will bind to the complementary Smad3 mRNA transcripts and prevent translation. Absolute complementarity, although preferred, is not required. A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have recently shown to be effective at inhibiting translation of mRNAs as well. See generally, Wagner, R., 1994, Nature 372:333-335. Thus, oligonucleotides complementary to either the 5′- or 3′-non-translated, non-coding regions of Smad3 could be used in an antisense approach to inhibit translation of endogenous Smad3 mRNA. Oligonucleotides complementary to the 5′ untranslated region of the mRNA should include the complement of the AUG start codon. Antisense oligonucleotides complementary to mRNA coding regions can also be used in accordance with the invention. Whether designed to hybridize to the 5′-, 3′- or coding region of Smad3 mRNA, antisense nucleic acids should be at least six nucleotides in length, and are preferably oligonucleotides ranging from 6 to about 50 nucleotides in length. In specific aspects the oligonucleotide is at least 6 nucleotides, at least 17 nucleotides, at least 25 nucleotides or at least 50 nucleotides.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide can include other appended groups such as peptides (e.g., for targeting host cell receptors in vivo), agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988) or other barriers, hybridization-triggered cleavage agents (See, e.g., Krol et al., 1988, BioTechniques 6:958-976) or intercalating agents (See, e.g., Zon, 1988, Pharm. Res. 5:539-549). To this end, the oligonucleotide can be conjugated to another molecule, e.g., a peptide, hybridization triggered cross-linking agent, transport agent, hybridization-triggered cleavage agent, etc.

The antisense oligonucleotide can comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide can also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

The oligonucleotides described herein can be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

The antisense molecules can be delivered to cells that express the Smad3 protein in vivo, e.g., sites of cutaneous wound healing. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation of endogenous mRNAs. Therefore, a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous Smad3 transcripts and thereby prevent translation of the Smad3 mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), etc. An epidermal specific promoter, such as a keratin based vector that has its expression induced by a variety of appropriate stimuli including wounding is desirable. Any type of plasmid, cosmid, YAC or viral vector can be used to prepare the recombinant DNA construct which can be introduced directly into the tissue site; e.g., the site of cutaneous wound healing. Alternatively, viral vectors can be used, which selectively infect the desired tissue; (e.g., for skin, papillomavirus vectors may be used), in which case administration may be accomplished by another route (e.g., systemically).

Ribozyme molecules-designed to catalytically cleave Smad3 mRNA transcripts can also be used to prevent translation of Smad3 mRNA and expression of Smad3. (See, e.g., PCT International Publication WO90/11364, published Oct. 4, 1990; Sarver et al., 1990, Science 247:1222-1225). While ribozymes that cleave mRNA at site specific recognition sequences can be used to destroy Smad3 mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591. There are a plurality of potential hammerhead ribozyme cleavage sites within the nucleotide sequence of human Smad3 cDNA. Preferably the ribozyme is engineered so that the cleavage recognition site is located near the 5′ end of the Smad3 mRNA; i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent-application No. WO 88/04300 by University Patents Inc.; Been and Cech, 1986, Cell, 47:207-216). The Cech-type ribozymes have an eight base pair active site, which hybridizes to a target RNA sequence whereafter cleavage of the target RNA takes place. Aspects of the invention encompass those Cech-type ribozymes that target eight base-pair active site sequences that are present in Smad3.

As in the antisense approach, the ribozymes can be composed of modified oligonucleotides (e.g. for improved stability, targeting, etc.) and should be delivered to cells which express Smad3 in vivo, e.g., sites of cutaneous wound healing. A preferred method of delivery involves using a DNA construct “encoding” the ribozyme under the control of a strong constitutive pol III or pol II promoter, so that transfected cells will produce sufficient quantities of the ribozyme to destroy endogenous Smad3 messages and inhibit translation. Because ribozymes unlike antisense molecules, are catalytic, a lower intracellular concentration is required for efficiency.

Endogenous Smad3 gene expression can also be reduced by inactivating or “knocking out” the Smad3 gene or its promoter using targeted homologous recombination. (E.g., see Smithies et al., 1985, Nature 317:230-234; Thomas & Capecchi, 1987, Cell 51:503-512; Thompson et al., 1989 Cell 5:313-321). For example, a mutant, non-functional Smad3 protein (or a completely unrelated DNA sequence) flanked by DNA homologous to the endogenous Smad3 gene (either the coding regions or regulatory regions of the Smad3 gene) can be used, with or without a selectable marker and/or a negative selectable marker, to transfect cells that express Smad3 in vivo. Insertion of the DNA construct, via targeted homologous recombination, results in inactivation of the Smad3 gene. This approach is acceptable for use in humans provided the recombinant DNA constructs are directly administered or targeted to the required site using appropriate viral vectors, e.g., papillomavirus vectors for in vivo delivery to sites of cutaneous wound healing, or retrovirus vectors for in vitro transduction of autologous skin grafts.

Alternatively, endogenous Smad3 gene expression can be reduced by targeting deoxyribonucleotide sequences complementary to the regulatory region of the Smad3 gene (i.e., the Smad3 promoter and/or enhancers) to form triple helical structures that prevent transcription of the Smad3 gene in target cells in the body. (See generally, Helene, C. 1991, Anticancer Drug Des., 6(6):569-84; Helene, C., et al., 1992, Ann, N.Y. Accad. Sci., 660:27-36; and Maher, L. J., 1992, Bioassays 14(12):807-15).

In yet another embodiment, the activity of Smad3 can be reduced using a “dominant negative” approach to protect against fibrosis and improve wound healing. To this end, constructs that encode defective Smad3 proteins, can be used in gene therapy approaches to diminish the activity of Smad3 in appropriate target cells. For example, nucleotide sequences that direct host cell expression of Smad3 in which a domain or portion of a domain is deleted or mutated can be introduced into cells at sites of high-dose radiation exposure or cutaneous wound healing (by gene therapy methods described above). Alternatively, targeted homologous recombination can be utilized to introduce such deletions or mutations into the subject's endogenous Smad3 gene at sites of high-dose radiation exposure or cutaneous wound healing. The engineered cells will express non-functional Smad3 (i.e., a Smad 3 that is capable of binding its natural ligand, but incapable of signal transduction). Such engineered cells at sites of high-dose radiation exposure or cutaneous wound healing should demonstrate a heightened response to TGF-β, resulting in protection against fibrosis and improved wound healing.

Pharmaceutical Preparations and Methods of Administration

The compounds that are determined to affect Smad3 gene expression or Smad3 activity can be administered to a patient at therapeutically effective doses to protect against fibrosis and improve wound healing. A therapeutically effective dose refers to that amount of the compound sufficient to result in protection against fibrosis and improvement of wound healing. The compounds of the invention are generally administered to animals, including humans.

Effective Dose

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

It will be appreciated that the actual preferred amounts of active compound in a specific case will vary according to the specific compound being utilized, the particular compositions formulated, the mode of application, and the particular situs and organism being treated. Dosages for a give host can be determined using conventional considerations, e.g., by customary comparison of the differential activities of the subject compounds and of a known agent, e.g., by means of an appropriate, conventional pharmacological protocol.

Formulation and Use

The pharmacologically active compounds of this invention can be processed in accordance with conventional methods of galenic pharmacy to produce medicinal agents for administration to patients, e.g., mammals including humans.

The compounds of this invention can be employed in admixture with conventional excipients, i.e., pharmaceutically acceptable organic or inorganic carrier substances suitable for parenteral, enteral (e.g., oral) or topical application, which do not deleteriously react with the active compounds. Suitable pharmaceutically acceptable carriers include but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds. They can also be combined where desired with other active agents, e.g., vitamins.

For parenteral application, particularly suitable are injectable, sterile solutions, preferably oily or aqueous solutions, as well as suspensions, emulsions, or implants, including suppositories. Ampoules are convenient unit dosages.

For enteral application, particularly suitable are tablets, dragees, liquids, drops, suppositories, or capsules. A syrup, elixir, or the like can be used wherein a sweetened vehicle is employed.

Sustained or directed release compositions can be formulated, e.g., by inclusion in liposomes or incorporation into an epidermal patch with a suitable carrier, for example DMSO. It is also possible to freeze-dry these compounds and use the lyophilizates obtained, for example, for the preparation of products for injection.

For topical application, there are employed as non-sprayable forms, viscous to semi-solid or solid forms comprising a carrier compatible with topical application and having a dynamic viscosity preferably greater than water. Suitable formulations include but are not limited to solutions, suspensions, emulsions, creams, ointments, powders, liniments, salves, aerosols, etc., which are, if desired, sterilized or mixed with auxiliary agents, e.g., preservatives, stabilizers, wetting agents, buffers or salts for influencing osmotic pressure, etc. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., a freon.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Smad3 Disruption Leads to Accelerated Wound Healing.

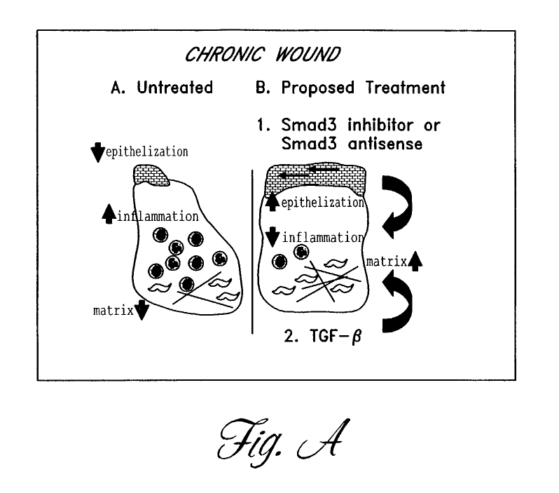

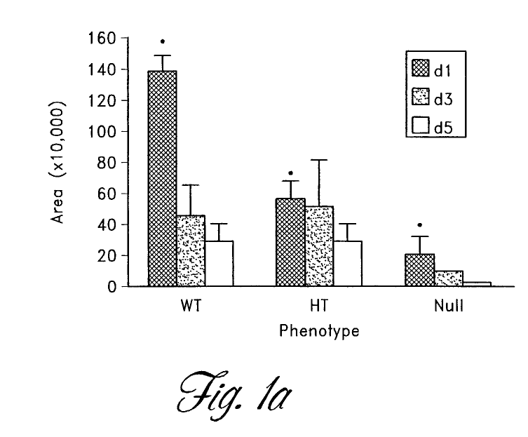

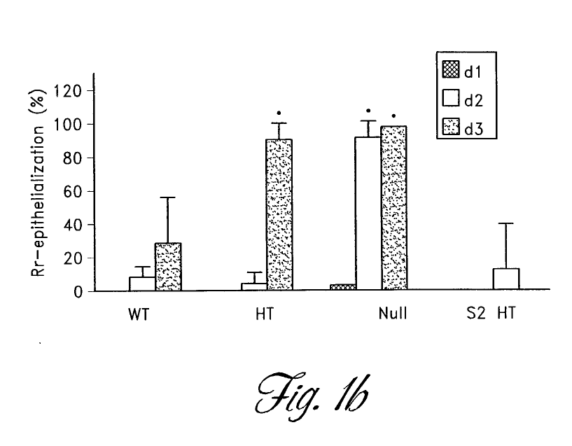

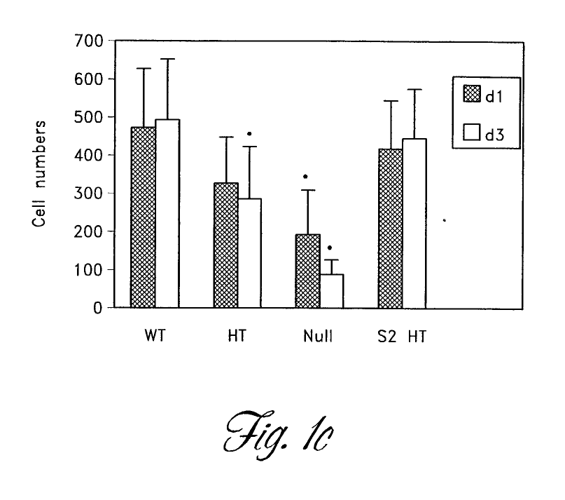

Following full-thickness incisional wounds (Ashcroft, G. S. et al. Estrogen accelerates cutaneous wound healing associated with an increase in TGF-beta 1 levels. Nature Med. 3, 1209-1215 (1997)), the rate of wound healing was markedly accelerated in healthy Smad3^{ex8/ex8} mice (Table 1), with complete re-epithelialization occurring by day 2 post-wounding in the null mice versus day 5 in the wild-type mice (FIG. 1b), and with significantly reduced wound areas (FIG. 1a) and wound widths visible. Total cell numbers (fibroblasts and inflammatory cells) were markedly reduced in the wounds of the Smad3^{ex8/ex8} mice, with intermediate numbers present in the heterozygous mice (FIG. 1c), compared with wild-type controls. Giemsa staining of sections in conjunction with immunostaining for a monocyte marker indicated that both neutrophils and monocytes were largely absent from the early wounds of Smad3^{ex8/ex8} mice. The wound areas of the Smad3^{ex8/ex8} mice were significantly smaller than those of wild-type mice, with reduced quantities of granulation tissue present at all time points. Wound contraction occurs through the relative contributions of re-epithelialization and myofibroblast action, and thus the accelerated re-epithelialization in the Smad3^{ex8/ex8} mice, and/or increased contractility of wound fibroblasts, presumably contribute to this phenotype. This observation corroborates earlier controversial studies indicating that central granulation tissue may not be critical to wound closure (Gross, J. et al. On the mechanism of skin wound “contraction”: a granulation tissue “knockout” with a normal phenotype. Proc. Natl Acad. Sci. USA 92, 5982-5986 (1995)).

TABLE 1
Accelerated wound healing after targeted Smad3 disruption

| Phenotype | Day 1 | Day 2 | Day 3 | Day 5 |
|---|---|---|---|---|
| Wild-type | Inflammation | No re- | No re- | Re- |
| | (+++) | Epitheliali- | Epithelia- | Epithelia- |
| | Wide wound | zation | lization | lized |
| | (++) | Granulation | | Moderate |
| | | tissue | | wound |
| | | (++) | | width |
| Smad3 | Inflammation | No re- | Re- | Moderate |
| Hetero- | (++) | epitheliali- | Epithelia- | wound |
| zygote | Wide wound | zation | lized | width |
| | (+) | Granulation | | |
| | | tissue | | |
| | | (++) | | |
| Smad3 | Reduced | Re- | Re- | Narrow |
| Knockout | Inflammation | epithelia- | Epithelia- | wound |
| | Narrow | lized | lized | width |
| | Wound | Reduced | | |
| | | granulation | | |
| | | tissue | | |

Effects Of Exogenous TGF-β on the Wound-Healing Response.

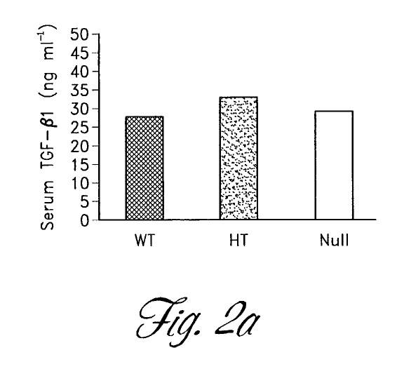

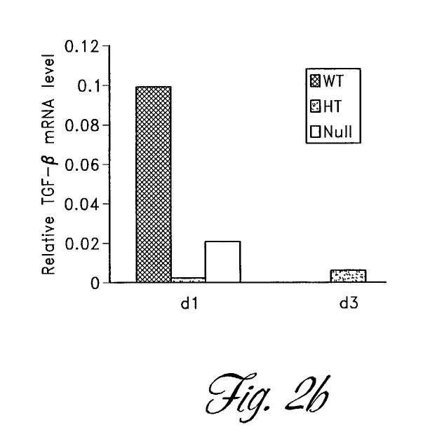

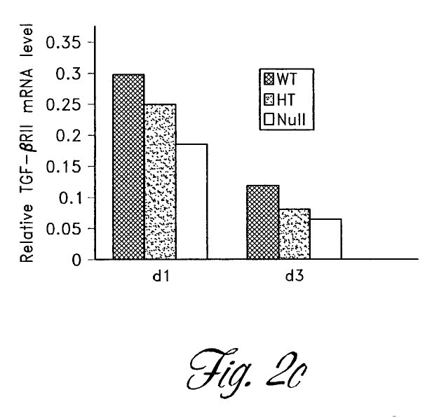

TGF-β released from degranulating platelets at wound sites has a broad spectrum of effects on, and is secreted by, each of the diverse cell types involved in wound healing. Specifically, these cells include the keratinocyte, responsible for reconstruction of the cutaneous barrier, the fibroblast, responsible for matrix production, and the monocyte, which infiltrates the wound at an early stage and secretes a vast array of cell-regulatory cytokines, including TGF-β (Roberts, A. B. TGF-beta: activity and efficacy in animal models of wound healing. Wound Repair Regen. 3, 408-418 (1995)); (O'Kane, S. & Ferguson, M. W. J. TGF-beta s and wound healing. Int. J. Biochem. Cell Biol. 29, 63-78 (1997)). As we observed a marked reduction in the number of monocytes in the wounds of the null mice, we proposed that part of the healing phenotype was secondary to the reduced levels of TGF-β, a potent monocyte chemoattractant, secreted by these inflammatory cells (Wahl, S. M. et al. Transforming growth factor type beta induces monocyte chemotaxis and growth factor production. Proc. Natl Acad. Sci. USA 84, 5788-5792 (1987)). Moreover, depletion of monocytes in animal models leads to a reduced fibrotic response, consistent with the role of these cells in TGF-β secretion (Leibovich, S. J. & Ross, R. The role of the macrophage in wound repair. A study with hydrocortisone and antimacrophage serum. Am. J. Pathol. 78, 71-100 (1975)); (McCartney-Francis, N., & Wahl, S. M. Transforming growth factor beta: a matter of life and death. J. Leuk. Biol. 55, 401-109 (1994)). Although TGF-β1 was present at equivalent levels in the serum of all animals, probably representing TGF-β1 released from platelet α-granules (FIG. 2a), the null mice showed reduced immunostaining for TGF-β isoforms in wound leukocytes and decreased TGF-β1 RNA levels, particularly at day 3 (FIG. 2b), supporting our hypothesis that a reduction in local TGF-β1 amounts contribute to the aberrant wound-healing phenotype of these mice.

To address this question, we applied topical TGF-β1 immediately before wounding. Following treatment with TGF-β1, inflammatory-cell numbers were increased in the heterozygote but not in the Smad3^{ex8/ex8} wounds, indicating that Smad3 may be critical for TGF-β-mediated chemotaxis. Despite a failure to influence monocyte recruitment, addition of TGF-β1 to the wounds of the Smad3^{ex8/ex8} mice increased matrix deposition, corroborating previous studies that showed that monocytes affect matrix deposition indirectly through the production of TGF-β1 (Pierce, G. F. et al. Transforming growth factor beta reverses the glucocorticoid-induced wound-healing deficit in rats: possible regulation in macrophages by platelet-derived growth factor. Proc. Natl. Acad. Sci. USA 86, 2229-2233 (1989)). Exogenous TGF-β1 stimulated matrix deposition, most notably in the null and heterozygous mice, without evidence of increasing fibroblast numbers, consistent with the idea that reduced local levels of TGF-β1 in the Smad3^{ex8/ex8} mice underlie the decreased matrix deposition in these animals. Moreover, these data indicate that expression of TGF-β receptors in the wounds of the null mice is adequate for matrix production. (FIG. 2c) The SMAD signaling pathway may be important for collagen expression, whereas fibronectin (matrix) synthesis may be induced by TGF-β through a c-Jun (SMAD-independent) pathway (Vindevoghel, L. et al. SMAD3/4-dependent transcriptional activation of the human type VII collagen gene (COL7A1) promoter by transforming growth factor beta. Proc. Natl Acad. Sci. USA 95, 14769-14774 (1998)); (Chen, S. J. et al. Stimulation of type I collagen transcription in human skin fibroblasts by TGF-beta: involvement of Smad3. J. Invest. Dermatol. 112, 49-57 (1999)); (Hocevar, B. A., Brown, T. L. & Howe, P. H. TGF-beta induces fibronectin synthesis through a c-Jun N-terminal Kinase-dependent, Smad4-independent pathway. EMBO J. 18, 1345-1356 (1999)). In agreement with this, our data also implicate a Smad3-independent pathway in early fibroblast matrix production in vivo.

Mechanisms Underlying a Reduced Local Monocyte Influx.

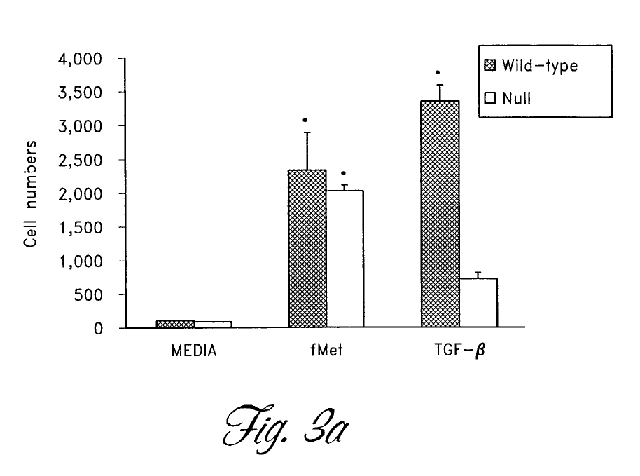

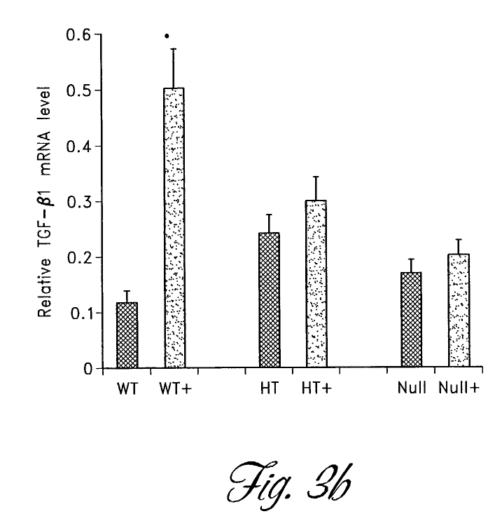

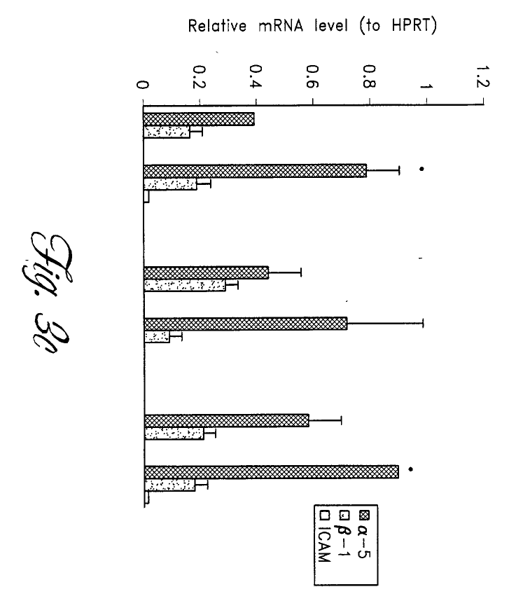

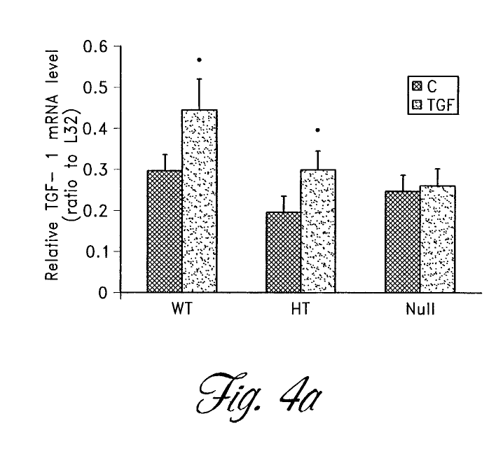

As Smad3 appeared to be potentially important in monocyte function, we focused on the mechanisms underlying these observations. If circulating monocytes are to infiltrate the sites of injury/inflammation, they must first respond to a local chemoattractant signal and traverse the endothelial basement membrane. TGF-β is a key factor in this response because, in vivo, femtomolar concentrations of TGF-β induce the most potent known chemoattractant response by circulating blood monocytes (Wahl, S. M. et al. Transforming growth factor type beta induces monocyte chemotaxis and growth factor production. Proc. Natl Acad. Sci. USA 84, 5788-5792 (1987)); (Wiseman, D. M., Polverini, P. J., Kamp, D. W. & Leibovich, S. J. Transforming growth factor-beta (TGF beta) is chemotactic for human monocytes and induces their expression of angiogenic activity. Biochem. Biophys. Res. Commun. 157, 793-800 (1988)). To investigate the mechanisms underlying the observed reduction in wound monocyte numbers, we determined the effects of Smad3 deletion on monocyte chemotaxis and on the expression of TGF-β-regulated cell-adhesion molecules potentially important in the trans-endothelial migration and adhesion of monocytes (Wahl, S. M., Allen, J. B., Weeks, B. S., Wong, H. L. & Klotman, P. E. Trandforming growth factor beta enhances integrin expression and type IV collagenase secretion in human monocytes. Proc. Natl Acad. Sci. USA 90, 4577-4581 (1993)). Cultured Smad3^{ex8/ex8} monocytes exhibited significantly reduced specific chemotaxis to TGF-β1, but migrated normally to the classical chemoattractant fMet-Leu-Phe (FMLP), a G-protein-mediated response (FIG. 3a). Smad3^{ex8/ex8} monocytes also showed a failure to upregulate TGF-β1 expression in an autocrine fashion (FIG. 3b) despite a TGF-β mediated increase in levels of TGF-β receptor II (TGF-pRII). The data indicate that regulation of TGF-β1 and its receptor may occur independently, with Smad3 being involved in induction of TGF-β1 expression and Smad3-independent pathways (such as those involving Smad2 or MAP kinase) regulating receptor expression. Smad3-independent events may also be involved in TGF-β-mediated expression of integrins by monocytes (FIG. 3c).

To test the hypothesis that the initial reduction in monocyte numbers observed in the wounds of the Smad3-null mice contributed to the wound-healing phenotype, we applied freshly extracted monocytes from wild-type mice to Smad3^{ex8/ex8} wounds. Direct addition of wild-type monocytes at the time of wounding has a similar effect to that of injection of TGF-β. That is, reduced matrix deposition in the wounds of the Smad3^{ex8/ex8} mice does not reflect impairment of the ability of Smad3^{ex8/ex8} fibroblasts to elaborate matrix proteins per se, but instead results from the reduced levels of TGF-β in the wounds of the Smad3^{ex8/ex8} mice (reduced TGF-β levels being themselves a direct result of the reduced monocytic infiltrate). Injection of neither monocytes nor TGF-β affected re-epithelialization, so these two effects—matrix deposition and re-epithelialization—can be distinguished. We suggest that the decrease in monocyte infiltration is related to a lack of response by Smad3^{ex8/ex8} monocytes to an initial TGF-β1 chemotactic signal, despite retention of the ability to respond in terms of integrin upregulation. These events subsequently lead to reduced local levels of TGF-β, a characteristic that is secondary not only to reduced cell numbers but also to an absence of autocrine induction of TGF-62 1.

Role of Smad3 in Wound Re-Epithelialization.

As re-epithelialization is critical to optimal wound healing, not only because of the reformation of a cutaneous barrier but also because of its role in wound contraction, we further investigated the effects of Smad3 disruption on this process. In vitro, the effects of TGF-β are paradoxical: integrin-mediated keratinocyte migration is enhanced whereas keratinocyte proliferation is inhibited (Zambruno, G. et al. Transforming growth factor-beta 1 modulates beta 1 and beta 5 integrin receptors and induces the de novo expression of the alpha v beta 6 heterodimer in normal human keratinocytes: implications for wound healing. J. Cell Biol. 129, 853-865 (1995). Moreover, studies of the role of exogenous TGF-β on re-epithelialization have generated conflicting results, depending upon the dosage, kinetics of administration, and model chosen (Mustoe, T. A., Pierce, G. F., Morishima, C. & Deuel, T. F. Growth factor-induced acceleration of tissue repair through direct and inductive activities in a rabbit dermal ulcer model. J. Clin. Invest 87, 694-703 (1991)); (Hebda, P. A. Stimulatory effects of transforming growth factor-beta and epidermal growth factor on epidermal cell outgrowth from porcine skin explant cultures. J. Invest. Dermatol. 91, 440-445 (1988)). Here, despite the presence of similar wound widths in the wild-type and heterozygous mice at day 3, complete re-epithelialization had occurred in the heterozygous mice by this time point, indicating that TGF-β signaling in vivo in keratinocytes is a Smad3-dependent process that ultimately leads to the inhibition of re-epithelialization. To evaluate the specificity of Smad3 in this signaling pathway, we also analyzed the wound-healing phenotype in Smad2 heterozygotes. Wounds of these mice heal to produce wound widths and areas that are similar to those seen in Smad3 heterozygotes and wild-type mice at day 3 (FIG. 1); however, in contrast to the Smad3 heterozygotes, wounds of Smad2 heterozygotes did not re-epithelialize (FIG. 1b). These results indicate that Smad3 may have effects on in vivo epithelial biology that are different to those of Smad2. Although Smad2 and Smad3 occasionally appear to function interchangeably when overexpressed in vitro, the unique abilities Smad3 to bind DNA directly and to interact with oncogenes such Evi-1 and nuclear receptors such as the vitamin D3 receptor indicate that these two SMADs may regulate distinct target genes in vivo (Yanagisawa, K. et al. Induction of apoptosis by Smad3 and down-regulation of Smad3 expression in response to TGF-beta in human normal lung epithelial cells. Oncogene 17, 1743-1747 (1998)); (Dennler, S., Huet, S. & Gauthier, J. M. A short amino—acid sequence in MH1 domain is responsible for functional differences between Smad2 and Smad3. Oncogene 18, 1643-1648 (1999)); (Ulloa, L., Doody, J. & Massague, J. Inhibition of transforming growth factor-beta/SMAD signaling by the interferon-gamma/STAT pathway. Nature 397, 710-713 (1999)); (Yanagisawa, J. et al. Convergence of transforming growth factor-beta and vitamin D signaling pathways on SMAD transcriptional coactivators. Science 283, 1317-1321 (1999)); (Kurokawa, M. et al. The oncoprotein Evi-1 represses TGF-beta signaling by inhibiting Smad3. Nature 2, 92-96 (1998)). This idea is supported by the striking differences in their respective null phenotypes (Yang, X. et al. Targeted disruption of SMAD3 results in impaired mucosal immunity and diminished T cell responsiveness to TGF-beta. EMBO J. 18, 1280-1291 (1999)); (Datto, M. B. et al. Targeted disruption of Smad3 reveals an essential role in transforming growth factor beta-mediated signal transduction Mol. Cell biol. 19, 2495-2504 (1999)); (Zhu, Y., Richardson, J. A., Parada, L. F., & Graff, J. M. Smad3 mutant mice develop metastatic colorectal cancer. Cell 18, 703-714 (1998)); (Weinstein, M., Yang, X., Li, C., Xu, X., & Deng. C. Failure of extraembryonic membrane formation and mesoderm induction in embryos lacking the tumor suppressor Smad2. Proc. Natl. Acad. Sci USA 95, 9378-9383 (1998)).

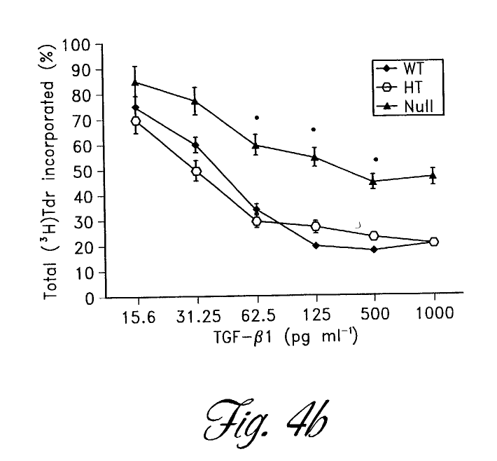

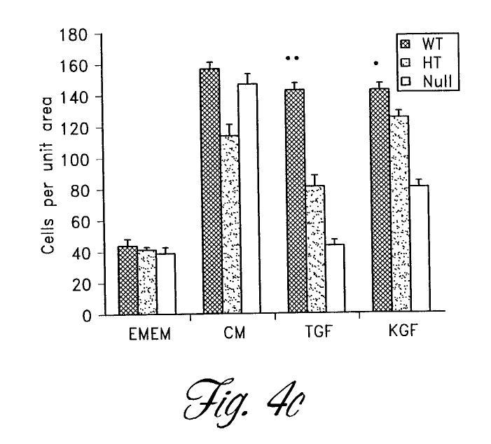

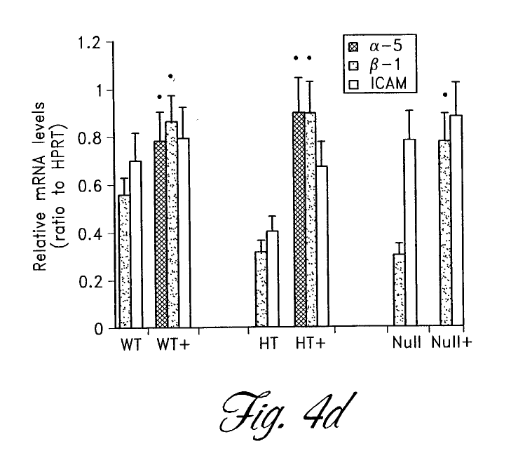

To identify the mechanisms underlying the in vivo effects of Smad3 on re-epithelialization, we tested whether keratinocyte functions crucial to wound repair, namely migration and proliferation, were modified by Smad3 disruption. Although expression levels of TGF-β receptors in keratinocytes were independent of the Smad3 genotype, Smad3^{ex8/ex8} keratinocytes lacked the ability to upregulate TGF-β expression in response to TGF-β1 (FIG. 4a). As Smad3 is involved in the inhibition of cell growth, we reasoned that enhanced re-epithelialization in the Smad3^{ex8/ex8} mice might be secondary to enhanced proliferative capacity (Datto, M. B. et al. Targeted disruption of Smad3 reveals an essential role in transforming growth factor beta-mediated signal transduction Mol. Cell biol. 19, 2495-2504 (1999)). In culture, primary keratinocytes derived from the Smad3-null mice showed a reduced sensitivity to growth inhibition by TGF-β (FIG. 4b). These findings were paralleled by an increase in basal keratinocyte proliferation (as judged by incorporation of bromodeoxyuridine (BrdU)) at the wound edge in the null cells compared with wild-type cells (FIG. 4b). The results show that high levels of exogenous TGF-β can inhibit the growth of the heterozygous and wild-type keratinocytes equally. However, we interpret the intermediate result in terms of re-epithelialization of cutaneous wounds in the heterozygous mice to result from the reduced level of endogenous TGF-β produced (compared with wild-type levels), as the inflammatory response is still blunted compared with the wild-type response.

A further aspect of re-epithelialization involves cell migration across matrix components in response to a chemoattractant gradient. Smad3^{ex8/ex8} keratinocytes exhibited reduced adhesion to matrix and migration towards TGF-β and keratinocyte growth factor (KGF), while maintaining a normal response towards growth factors present in conditioned media (FIG. 4c). An increasing number of cytokines and alternative signaling pathways have been shown to affect SMAD activity (Ulloa, L., Doody, J. & Massague, J. Inhibition of transforming growth factor-beta/SMAD signaling by the interferon-gamma/STAT pathway. Nature 397, 710-713 (1999)); (Yanagisawa, J. et al. Convergence of transforming growth factor-beta and vitamin D signaling pathways on SMAD transcriptional coactivators. Science 283, 1317-1321 (1999)); (Kurokawa, M. et al. The oncoprotein Evi-1 represses TGF-beta signaling by inhibiting Smad3. Nature 2, 92-96 (1998)); (de Caestecker, M. P. et al. Smad2 transduces common signals from receptor serine-threonine and tyrosine kinases. Genes Dev. 12, 587-592 (1998)), so it is possible that KGF may mediate some of its effects on wild-type cells through interplay with the Smad3 signaling pathway. Because integrins are pivotal in mediating cell migration, we reasoned that Smad3 may be required for TGF-β-induced integrin expression by keratinocytes. Exogenous TGF-β1 upregulated expression of β₁ integrins but not of the α₅ subunit in the null cells; this may represent an underlying mechanism for impaired migration across fibronectin (FIG. 4d). This effect differs from that of altered Smad3 signaling in the monocyte, indicating that the effects of Smad3 disruption on a particular gene target depend on the cellular context and cannot be generalized. We also assessed the effect of Smad3 disruption on cell-adhesion molecules specific to keratinocytes, namely E-cadherin and syndecan-1. The expression levels of both were equivalent in all phenotypes, both basally and following TGF-β treatment. Thus, in the context of wound healing, one possible mechanism of enhanced re-epithelialization in the Smad3^{ex8/ex8} mice may involve increased keratinocyte proliferation (compared with wild-type keratinocytes) in the presence of TGF-β, coupled with a migratory response stimulated by growth factors other than TGF-β and KGF in a Smad3-independent process. These data indicate the importance of the early proliferative response in accelerating in vivo re-epithelialization, which appears to be inhibited by a Smad3-dependent pathway.

Smad3 Disruption Leads To Protection Against Radiation-Induced Fibrosis.

Male wildtype or Smad3 null littermates, 6 weeks of age, were exposed to radiation on the right thigh region. The left leg served as an internal control. In this initial experiment, mice were either not radiated, or given 30 or 60 Gy in a single dose. Mice were killed at 2 weeks and 5 weeks post-radiation. Tatoo marks 1 cm apart were used to assess contraction of the skin and a torsion test was used to measure contractility of the leg. Sections of the skin and muscle were fixed in neutral buffered formalin for histology.

Analysis of the histology of the skin at 2 weeks post-radiation demonstrated that the skin of Smad3 null mice is resistant to the damaging effects of radiation. Comparison of the non-radiated skin of the left thigh of wildtype mice and the skin of the right thigh that received 60 Gy radiation showed a severe hyperplasia of the epidermis and hair follicles resulting from this high dose of radiation. In contrast, there was only the mildest hyperplasia in the skin of the radiated thigh of the Smad3 null mice, and the hair follicles looked normal. The area of compacted connective tissue (scar) had a greater area in the radiated wildtype compared to the Smad3 null skin. The inflammatory response was also stronger in the wildtype mice. These data establish that Smad3 plays an essential role in the response of epidermal/dermal hair follicle cells to radiation damage and that cells lacking Smad3 are resistance to radiation-induced injury.

Pictures were taken of the radiated right thighs of littermate wildtype or Smad3 null male mice 5 weeks post-exposure to a single 60 Gy dose of radiation. The skin of the wildtype mice was thickened, contracted (as measured by the distance of the two tatoo marks) and lacking regrown hair over the radiated area. In striking contrast, the skin of the Smad3 null mice had retained normal flexibility, pigment, and showed regrowth of hair over the radiated area. These observations support the conclusion that loss of Smad3 prevents the long-term effects of high-dose radiation, such as fibrosis, scarring, and alopecia.

Histology was analyzed of the skin of the radiated right thighs of littermate wildtype or Smad3 null male mice 5 weeks post-exposure to a single 60 Gy dose of radiation. The two wildtype and two Smad3 null mice examined showed a variable response condition. Nevertheless, patterns could be discerned. On average, the degree of epidermal hyperplasia was significantly higher in the wildtype mice. Additionally, the area of mild hyperplasia in the Smad3 null mice was quite limited, whereas in the wildtype mice the area of epidermal involvement was quite extensive and uniform. These observations further support the conclusion that Smad3 disruption leads to protection against radiation-induced fibrosis.

### EXAMPLE

Wound-Healing Experiments.

Smad3^{ex8/ex8} mice were generated by targeted disruption of the Smad3 gene by homologous recombination. Targeted embryonic-stem-cell clones were injected into germline transmission. Mice heterozygous for the targeted disruption were intercrossed to produce homozygous offspring (Yang, X. et al. Targeted disruption of SMAD3 results in impaired mucosal immunity and diminished T cell responsiveness to TGF-beta. EMBO J. 188, 1280-1291 (1999)). 48 4-6-week-old mice (Smad3 wild-type, heterozygotes and null mice) were anaesthetized with methoxyfluorane, and the dorsum was shaved and cleaned with alcohol. Four equidistant 1-cm full-thickness incisional wounds were made through the skin and panniculus carnosus muscle. For a subset of animals, before wounding, the area to be incised was injected subcutaneously with 50 μl of either vehicle (PBS+4 mM HCI) or TGF-β1 (1 μg), or was left unmanipulated. Treatments were rotated to ensure no site bias. Wounds were collected at days 1, 2, 3 and 5 post-wounding and were bisected for histology and immunostaining, or snap-frozen in liquid nitrogen for RNA analysis. In addition, ten healthy Smad2 heterozygote mice (aged 4-6 weeks) underwent 1-cm incisional wounds as described, with wound excision at day 3 or 5. For analysis of BrdU incorporation, 150 mg kg⁻¹ BrdU solution (Sigma) was injected intraperitoneally 1 h before the mice were killed, and tissues were stained with monoclonal mouse anti-BrdU antibody (DAKO). Serum levels of TGF-β1 were measured using a Quantikine kit (R&D systems).

Histology, Immunocytochemistry and Image Analysis.

Histological sections were prepared from wound tissue fixed in 10% buffered formal saline and embedded in paraffin. 7-μm sections were stained with haematoxylin and eosin, Masson's trichrome or Giemsa, or were subjected to immunohistochemistry with antibodies to TGF-β 1, 2 and 3 (Santa Cruz) or fibronectin, used at a dilution of 1:20 in PBS. Image analysis and quantification of cell numbers per unit area, of wound area (measured below the clot and above the panniculus muscle) and of re-epithelialization was done using an Optimas program as described (Ashcroft, G. S. et al. Estrogen accelerates cutaneous wound healing associated with an increase in TGF-beta 1 levels. Nature Med. 3, 1209-1215 (1997)).

Culture of Bone-Marrow Monocytes and Chemotaxis Assay.

Bone marrow was collected from the femurs and tibias of 4-6-week-old male mice. Mononuclear cells were isolated using a two-component step gradient (Cardinal Associates Inc., Santa Fe), and incubated for 4-7 days in monocyte colony-stimulating factor (10 ngml⁻¹) as described (Feldman, G. et al., STAT5A-deficient mice demonstrate a defect in granulocyte-macrophage colony-stimulating factor-induced proliferation and gene expression. Blood. 90, 1768-1776 (1997)). Chemotaxis of monocytes was stimulated in a 12-well chemotaxis chamber (Corning Costar Transwell Plate), in triplicate wells containing 400 ml FMLP (1 μM), control media, or TGF-β (1 pgml⁻¹). Monocytes were resuspended in chemotaxis buffer (Hank's buffer with 0.5% BSA) at a final concentration of 3×10⁵ per 100 μl; 100 μl was added to the upper chamber, and the monocytes were incubated for 90 min at 37° C. in a humidified atmosphere (5% CO₂). Cells that migrated across the membrane (pore size 3 μm) were fixed in 40 μl chemotaxis fixative (100 mM EDTA and 10% formaldehyde in PBS) and counted in 500-μl volume using a Coulter counter. For wound-healing experiments using monocytes, bone-marrow monocytes removed from wild-type mice were resuspended in PBS and 0.5×10⁶ cells (or PBS vehicle alone) were injected subcutaneously at the site to be incised. Immediately after injection, 1-cm full-thickness incisions were made (as above) and the wounds excised at day 3 post-wounding.

Keratinocyte Adhesion/Migration and Proliferation Assays.

Keratinocytes were isolated from the skin of newborn mice from crosses of Smad3 heterozygote adults by standard methods (Dlugosz, A. A., Glick, A. B., Tennenbaum, T., Weinberg, W. C. & Yuspa, S. H. Isolation and utilization of epidermal keratinocytes for oncogene research. Methods Enzymol. 254, 3-20 (1995)). Cells were plated in EMEM medium, 8% chelexed fetal bovine serum, 0.2 mM CaCl₂ with antibiotics, and then switched to the same media with 0.05 mM CaCl₂. For migration assays, cells were trypsinized, washed and resuspended to 1×10⁶ cells ml⁻¹ in serum-free EMEM. 5×10⁴ cells were added to the upper well of a chemotaxis chamber (Neuro Probe Inc.); this upper well was separated from the test medium (which was EMEM, conditioned medium from wild-type keratinocytes, KGF or TGF-β1 at 1 ng ml⁻¹) in the lower chamber by a fibronectin/collagen-I-coated membrane. Cells that had migrated through the membrane after 5 h at 37° C. were stained using Diff-Quick and counted from video images obtained with a Leitz photomicroscope. Each value represents the average number of cells migrated from triplicate wells. For proliferation assays, cells were seeded at 80,000 cells per well in a 24-well tissue-culture tray and allowed to proliferate for 3 days. Porcine TGF-β1 (R&D Systems) was added at varying concentrations for 20 h and the wells were pulsed with 1 μCi [³H]thymidine for an extra 4 h. Radioactivity incorporated into DNA was determined by established methods (Danielpour, D. et al. Immunodetection and quantitation of the two forms of transforming growth factor-beta (TGF-beta 1 and TGF-beta 2) secreted by cells in culture. J. Cell Physiol. 138, 79-86 (1989)). Each value represents the average of triplicate wells.

Expression of Cell-Adhesion Molecules and TGF-β Isoforms.

Wound tissue (microdissected to avoid contamination from unwounded adjacent skin) and normal skin from the dorsal area were homogenized and total RNA was extracted with trizol. In addition, total RNA was extracted in a similar fashion from monocytes and keratinocytes. Reverse transcription with polymerase chain reaction was done using the following primers (band intensities were normalized to those of the keratinocyte/monocyte housekeeping gene HPRT (hypoxanthine phosphoribosyl transferase); α, integrin, 5′-CATTTCCGAGTCTGGGCCA (SEQ ID NO:3) and 5′-TGGAGGCTTGAGCTGAGCTT (SEQ ID NO:4); β₁, integrin, 5′-TGTTCAGTGCAGAGCCTTCA (SEQ ID NO:5) and 5′-CCTCATACTTCGGATTGACC (SEQ ID NO:6); intercellular adhesion molecule (ICAM), 5′-TTCMCCCGTGCCAAGCCCACGCT (SEQ ID NO:7) and 5′-GCCAGCACCGTGAATGTGATCTCC (SEQ ID NO:8); E-cadherin, 5′-TCAGCACCCACACACATACA (SEQ ID NO:9) and 5′-GCATTTTCTCAGGMGCAGG (SEQ ID NO:10); syndecan-1, 5′-GATCCCAAAGCCACTGTGTT (SEQ ID NO:11) and 5′-ACACTGTGGMCCAGCCTTC (SEQ ID NO: 12). In addition, RNase-protection assays were done according to the manufacturer's instructions (Pharmingen) using multiprobe templates on 3 μg total RNA, and were developed using phosphorimaging. Band densities were normalized to those of the keratinocyte monocyte housekeeping gene L32 for both the cytokine and the receptor templates, using an image-analysis program (Image Quant, Molecular Dynamics). All data were analyzed by Student's t-test or analysis of variance.

Although the invention has been described with reference to embodiments and examples, it should be understood that various modifications can be made without departing from the spirit of the invention. Accordingly, the invention is limited only by the following claims. All references cited herein are hereby expressly incorporated by reference.

What is claimed is:

1. A method of improving wound healing comprising the steps of:
a) administering a Smad3 inhibitor to a patient in need thereof; and
b) measuring improvement of wound healing.

2. The method of claim 1, wherein said Smad3 inhibitor is a peptide.

3. The method of claim 1, wherein said Smad3 inhibitor is an antisense molecule.

4. The method of claim 1, wherein said Smad3 inhibitor is a ribozyme.

5. A method of improving wound healing comprising the steps of:
a) identifying a patient in need of improvement of wound healing; and
b) administering a Smad3 inhibitor to said patient in need thereof.

6. The method of claim 5, wherein said Smad3 inhibitor is a peptide.

7. The method of claim 5, wherein said Smad3 inhibitor is an antisense molecule.

8. The method of claim 5, wherein said Smad3 inhibitor is a ribozyme.

9. A method of preventing fibrosis comprising the steps of:
a) administering a Smad3 inhibitor to a patient in need thereof; and
b) measuring protection against fibrosis.

10. The method of claim 9, wherein said Smad3 inhibitor is a peptide.

11. The method of claim 9, wherein said Smad3 inhibitor is an antisense molecule.

12. The method of claim 9, wherein said Smad3 inhibitor is a ribozyme.

13. A method of preventing fibrosis comprising the steps of:
a) identifying a patient in need of protection against fibrosis; and
b) administering a Smad3 inhibitor to said patient in need thereof.

14. The method of claim 13, wherein said Smad3 inhibitor is a peptide.

15. The method of claim 13, wherein said Smad3 inhibitor is an antisense molecule.

16. The method of claim 13, wherein said Smad3 inhibitor is a ribozyme.

17. A method of identifying a compound that can be used to improve wound healing comprising the steps of:
a) administering a test Smad3 inhibitor to a subject; and
b) measuring the effect on wound healing, wherein a compound is selected as a candidate on the basis of improvement of wound healing.

18. The method of claim 17, wherein said Smad3 inhibitor is a peptide.

19. The method of claim 17, wherein said Smad3 inhibitor is an antisense molecule.

20. The method of claim 17, wherein said Smad3 inhibitor is a ribozyme.

21. A method of identifying a compound that can be used to protect against fibrosis comprising the steps of:
a) administering a test Smad3 inhibitor to a subject; and
b) measuring the effect on fibrosis, wherein a compound is selected as a candidate on the basis of protection against fibrosis.

22. The method of claim 21, wherein said Smad3 inhibitor is a peptide.

23. The method of claim 21, wherein said Smad3 inhibitor is an antisense molecule.

24. The method of claim 21, wherein said Smad3 inhibitor is a ribozyme.

25. A method of making a pharmaceutical comprising the step of combining the compound identified by claim 17 with a pharmaceutical carrier.

26. A method of making a pharmaceutical comprising the step of combining the compound identified by claim 21 with a pharmaceutical carrier.


SEQUENCE LISTING (12 sequences)

SEQ ID NO: 1 - DNA, 2303 bp, Homo sapiens
cccggcgtcccgtcgagcccagccccgccgggggcgctcctcgccgcccgcacgccctccccagccatgtcgtccatcctgcctttcactcccccgatcgtgaagcgcctgctgggctggaagaagggcgagcagaacgggcaggaggagaaatggtgcgagaaggcggtcaagagcctggtcaagaaactcaagaagacggggcagctggacgagctggagaaggccatcaccacgcagaacgtcaacaccaagtgcatcaccatccccaggtccctggatggccggttgcaggtgtcccatcggaaggggctccctcatgtcatctactgccgcctgtggcgatggccagacctgcacagccaccacgagctgcgggccatggagctgtgtgagttcgccttcaatatgaagaaggacgaggtctgcgtgaatccctaccactaccagagagtagagacaccagttctacctcctgtgttggtgccacgccacacagagatcccggccgagttccccccactggacgactacagccattccatccccgaaaacactaacttccccgcaggcatcgagccccagagcaatattccagagaccccaccccctggctacctgagtgaagatggagaaaccagtgaccaccagatgaaccacagcatggacgcaggttctccaaacctatccccgaatccgatgtccccagcacataataacttggacctgcagccagttacctactgcgagccggccttctggtgctccatctcctactacgagctgaaccagcgcgtcggggagacattccacgcctcgcagccatccatgactgtggatggcttcaccgacccctccaattcggagcgcttctgcctagggctgctctccaatgtcaacaggaatgcagcagtggagctgacacggagacacatcggaagaggcgtgcggctctactacatcggaggggaggtcttcgcagagtgcctcagtgacagcgctatttttgtccagtctcccaactgtaaccagcgctatggctggcacccggccaccgtctgcaagatcccaccaggatgcaacctgaagatcttcaacaaccaggagttcgctgccctcctggcccagtcggtcaaccagggctttgaggctgtctaccagttgacccgaatgtgcaccatccgcatgagcttcgtcaaaggctggggagcggagtacaggagacagactgtgaccagtaccccctgctggattgagctgcacctgaatgggcctttgcagtggcttgacaaggtcctcacccagatgggctccccaagcatccgctgttccagtgtgtcttagagacatcaagtatggtaggggagggcaggcttggggaaaatggccatacaggaggtggagaaaattggaactctactcaacccattgttgtcaaggaagaagaaatctttctccctcaactgaaggggtgcacccacctgttttctgaaacacacgagcaaacccagaggtggatgttatgaacagctgtgtctgccaaacacatttaccctttggccccactttgaagggcaagaaatggcgtctgctctggtggcttaagtgagcagaacaggtagtattacaccaccggcaccctccccccagactctttttttgagtgacagctttctgggatgtcacagtccaaccagaaacgcccctctgtctaggactgcagtgtggagttcaccttggaagggcgttctaggtaggaagagcccgcacgatgcagacctcatgcccagctctctgacgcttgtgacagtgcctcttccagtgaacattcccagcccagccccgccccgttgtgagctggatagacttgggatggggagggagggagttttgtctgtctccctcccctctcagaacatactgattgggaggtgcgtgttcagcagaacctgcacacaggacagcgggaaaaatcgatgagcgccacctctttaaaaactcacttacgttgtcctttttcactttgaaaagttggaaggactgctgaggcccagtgcatatgcaatgtatagtgtctattatcacattaatctcaaagagattcgaatgacggtaagtgttctcatgaagcaggaggcccttgtcgtgggatggcatttggtctcaggcagcaccacactgggtgcgtctccagtcatctgtaagagcttgctccagattctgatgcatacggctatattggtttatgtagtcagttgcattcattaaatcaactttatcatatgctcaaaaaaaaaaaaaag

SEQ ID NO: 2 - protein, 425 aa, Homo Sapiens
MSSILPFTPPIVKRLLGWKKGEQNGQEEKWCEKAVKSLVKKLKKTGQLDELEKAITTQNVNTKCITIPRSLDGRLQVSHRKGLPHVIYCRLWRWPDLHSHHELRAMELCEFAFNMKKDEVCVNPYHYQRVETPVLPPVLVPRHTEIPAEFPPLDDYSHSIPENTNFPAGIEPQSNIPETPPPGYLSEDGETSDHQMNHSMDAGSPNLSPNPMSPAHNNLDLQPVTYCEPAFWCSISYYELNQRVGETFHASQPSMTVDGFTDPSNSERFCLGLLSNVNRNAAVELTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLDKVLTQMGSPSIRCSSVS

SEQ ID NO: 3 - DNA, 19 bp, Artificial Sequence
  primer
catttccgagtctgggcca

SEQ ID NO: 4 - DNA, 20 bp, Artificial Sequence
  primer
tggaggcttgagctgagctt

SEQ ID NO: 5 - DNA, 20 bp, Artificial Sequence
  primer
tgttcagtgcagagccttca

SEQ ID NO: 6 - DNA, 20 bp, Artificial Sequence
  primer
cctcatacttcggattgacc

SEQ ID NO: 7 - DNA, 24 bp, Artificial Sequence
  primer
ttcaacccgtgccaagcccacgct

SEQ ID NO: 8 - DNA, 24 bp, Artificial Sequence
  primer
gccagcaccgtgaatgtgatctcc

SEQ ID NO: 9 - DNA, 20 bp, Artificial Sequence
  primer
tcagcacccacacacataca

SEQ ID NO: 10 - DNA, 20 bp, Artificial Sequence
  primer
gcattttctcaggaagcagg

SEQ ID NO: 11 - DNA, 20 bp, Artificial Sequence
  primer
gatcccaaagccactgtgtt

SEQ ID NO: 12 - DNA, 20 bp, Artificial Sequence
  primer
acactgtggaaccagccttc